(19) United States
(12) Patent Application Publication
(10) Pub. No.: US 2014/0356331 A1
(43) Pub. Date: Dec. 4, 2014

(54) INJECTABLE CNS-DERIVED ECM FOR TISSUE RECONSTRUCTION

(71) Applicants: Stephen F. Badylak, Pittsburgh, PA (US); Bryan N. Brown, Pittsburgh, PA (US); Peter M. Crapo, Warwick, RI (US); Scott A. Johnson, Allison Park, PA (US); Christopher J. Medberry, Pittsburgh, PA (US)

(72) Inventors: Stephen F. Badylak, Pittsburgh, PA (US); Bryan N. Brown, Pittsburgh, PA (US); Peter M. Crapo, Warwick, RI (US); Scott A. Johnson, Allison Park, PA (US); Christopher J. Medberry, Pittsburgh, PA (US)

(73) Assignee: UNIVERSITY OF PITTSBURGH-OF THE COMMONWEALTH SYSTEM OF HIGHER EDUCATION, Pittsburgh, PA (US)

(21) Appl. No.: 14/131,286

(22) PCT Filed: Jul. 6, 2012
(86) PCT No.: PCT/US12/45682

§ 371 Date: Aug. 20, 2014

Related U.S. Application Data
(60) Provisional application No. 61/505,655, filed on Jul. 8, 2011.

(51) Int. Cl. A61L 27/36; A61L 27/54; A61L 27/40

(52) U.S. Cl. CPC A61L 27/3687; A61L 27/40; A61L 27/54; A61L 2430/34
USPC 424/93.7; 435/268

(57) ABSTRACT

Methods useful for preparing central nervous system (CNS) derived extracellular matrix (ECM) materials, including powders and gels are provided. Also provided are CNS ECM preparations prepared according to those methods, and methods of treating a patient or mammal with CNS injury.

### CROSS REFERENCE TO RELATED APPLICATIONS

This application claims the benefit of U.S. Provisional Application No. 61/505,655, filed Jul. 8, 2011, entitled “CNS-Derived ECM for Tissue Reconstruction (Injectable Extracellular Matrix Gel for Targeted Tissue Regeneration),” which is incorporated herein by reference in its entirety.

The Sequence Listing associated with this application is filed in electronic format via EFS-Web and is hereby incorporated by reference into the specification in its entirety. The name of the text file containing the Sequence Listing is 6527_{—}121916_ST25.txt. The size of the text file is 1,222 bytes, and the text file was created on Jul. 2, 2012.

### BACKGROUND

Biologic materials composed of extracellular matrix (ECM) are increasingly used for tissue reconstruction and the surgical restoration of many tissues. While there are a number of commercially-available ECM preparations, such as urinary bladder matrix (UBM) and small intestinal submucosa (SIS) preparations (see below), those preparations are not necessarily effective in all circumstances.

While there is intense interest in regenerative medicine strategies for damaged CNS, development of effective ECM preparations that support regeneration of CNS tissue has proven to be challenging. Optimally, a CNS ECM preparation is an easily deliverable substrate that fills space (e.g., prevent collapse of CNS tissue following surgical excision or injury), controls bleeding, recruits stem cells, and serves a substrate for regenerative growth into the new substrate. From a commercial standpoint, the CNS ECM preparation would be easily and inexpensively manufactured, and easy to package and transport. Such a product would provide a highly desired and valuable tool for researchers of CNS regeneration, as well as a therapeutic tool for medical intervention.

### SUMMARY

Methods of preparing CNS tissue-derived ECM preparations (CNS ECM) for use in tissue regeneration, replacement, and healing in the CNS are disclosed herein along with CNS ECM preparations and methods of use of the CNS ECM preparations. These tissue-specific ECM preparations are shown herein to be more effective than non-tissue-specific ECM preparations, such as UBM ECM preparations, in nerve cell growth/propagation studies. The CNS ECM is derived from spinal cord and/or brain tissue, and in one embodiment, dura mater is removed from the brain or spinal cord tissue prior to processing is that the CNS ECM preparation is substantially free of dura mater-derived material. In two embodiments described herein, spinal cord-derived and whole brain-derived ECM preparations are referred to as spinal cord matrix (SCM) and whole brain matrix (WBM or B-ECM), respectively. The disclosed CNS ECM can be manufactured using robust and inexpensive enzymatic, chemical, and mechanical steps that would translate well to industrial scaling, packaging and distribution as a gel or powder. In certain embodiments, the CNS ECM preparations are gels. The various physical properties of gels allow for direct minimally invasive delivery, e.g. by injection, into injured tissues such as the brain. The disclosed gels create a local niche/microenvironment conducive to cell/tissue regeneration. Because the products described herein are to be used in live organisms and patients, the product is preferably not cytotoxic and would integrate well with surrounding tissue.

In certain embodiments, a CNS ECM preparation is lyophilized, freeze-dried or otherwise dehydrated for packaging, storage and delivery. Dried CNS ECM preparations can be comminuted, e.g., in a classification mill, to a desirable size, for example to less than about 1 mm or in one embodiment to less than about 840 microns (μm). In other embodiments, dehydrated or lyophilized CNS ECM preparation is reconstituted by hydration and solubilized to produce either a solution or a gel (e.g., hydrogel) for use. In various embodiments, CNS ECM preparations are sterilized using irradiation, ethylene oxide, or other methods.

In yet other embodiments, CNS ECM preparations can also be chemically modified to act as a cell or drug delivery system at a site of CNS injury or disease. For example, anti-tumor agents or GABA receptor agonists could be delivered for the treatment of certain cancers or anxiety and seizure associated ailments. Drug delivery can also include certain pharmaceuticals, such as acetylcholinesterase inhibitors, pain killers, or antispasmodics. Cell delivery can include neural stem or progenitor cells or various differentiated cell types.

CNS ECM preparations are particularly useful for inducing, supporting, and guiding the growth of neuronal cells into sites of CNS disease or injury. For example in one embodiment, a CNS ECM preparation is implanted or injected, around or near a site in need of wound healing, tissue remodeling, and/or tissue regeneration. In another non-limiting embodiment, such a CNS ECM preparation comprises cells. For example and without limitation, such a method comprises adding cells to a CNS ECM preparation in vitro and implanting or injecting the material at the damaged site. In yet another non-limiting embodiment, the CNS ECM preparation comprises bioactive or therapeutic agents, such as, without limitation growth factors, antibiotics, and anti-inflammatory agents. For example, in certain embodiments, CNS ECM preparations can be seeded with stem/progenitor cells and/or ensheathing glia (such as olfactory ensheathing glia, oligodendrocyte lineage cells and Schwann cells).

### BRIEF DESCRIPTION OF THE DRAWINGS

FIG. 1A-D are photomicrographs of unprocessed CNS tissues and CNS-derived ECM shown with hematoxylin and eosin (H&E) staining. Magnification 10×.

FIGS. 2A-B are bar graph representations showing the absence of DNA in decellularized CNS-derived ECM.

FIG. 3A-I are bar graph representations showing chemotaxis of human neural stem cell lines versus the concentration of ECM in various CNS ECM preparation.

FIG. 4A-I are bar graph representations showing mitogenesis of human neural stem cell lines versus the concentration of ECM in various CNS ECM preparation.

FIG. 5A-B are fluorescent photomicrographs showing neurite formation following treatment with a CNS-derived ECM gel. Cells were suspended in spinal cord ECM gel at 2.75 mg/ml submerged in reduced modified medium without growth factors. After 6, 12, or 24 days medium was removed from wells and 10 μM calcein AM was added to effect intracellular fluorescence prior to imaging.

FIG. 6A-D are bar graph representations showing growth factor concentrations spinal cord ECM and urinary bladder ECM as compared to native tissues.

FIG. 7 are graphs illustrating gel formation of CNS-derived ECM as a measure of viscosity versus time.

FIG. 8 shows a 4 week functional assessment of rats after spinal cord injury and treatment with ECM gel delivered to the site of injury.

FIGS. 9A and 9B are graphs showing collagen and sulfated glycosaminoglycan (sGAG) composition in B-ECM and SC-ECM scaffolds. (FIG. 9A)SC-ECM contains a significantly higher percentage of collagen than B-ECM. (FIG. 9B) B-ECM contains a significantly higher concentration of sGAGs than SC-ECM.

FIG. 10 shows SEM micrographs of B-ECM, SC-ECM and UBM-ECM gels at 1000× (A-C), and 10,000× (D-F), respectively. Photographs of B-ECM, SC-ECM and UBM-ECM gels are provided (G-I).

FIG. 11 provides multiphoton micrographs for B-ECM and SC-ECM, respectively.

FIGS. 12A and 12B are graphs showing an example absorbance curve (FIG. 12A) and Turbidimetric absorbance for B-ECM, SC-ECM and UBM over time as the pre-gel digests gel (FIG. 12B). FIGS. 12C-12F are graphs providing t_{tag}, t_{1/2}, t₉₅ and S for the gels (from left to right for each of the graphs of FIGS. 12C-12F: UBM-ECM, B-ECM, and SC-ECM).

FIG. 13 are graphs showing Sigmoidal storage and loss moduli curves for B-ECM, SC-ECM and UBM representing rheologic gelation kinetics at 8 mg/mL (FIG. 13A), 6 mg/mL (FIG. 13B), and 4 mg/mL (FIG. 13C. FIG. 13D is a graph depicting ultimate storage modulus for each of UBM-ECM, B-ECM, and SC-ECM at each concentration.

FIG. 14 are photomicrographs depicting the result of a live/dead assay for B-ECM (A) and SC-ECM (B).

FIG. 15 are graphs depicting % cells with neurite extension and Average neurite length for UBM-ECM (first row), B-ECM (second row) and SC-ECM (third row). Photomicrographs showing neurite extension are provided (bottom row).

FIG. 16 are photomicrographs showing three-dimensional neurite extensions for N1E-115 cells.

### DETAILED DESCRIPTION

The use of numerical values in the various ranges specified in this application, unless expressly indicated otherwise, are stated as approximations as though the minimum and maximum values within the stated ranges are both preceded by the word “about”. In this manner, slight variations above and below the stated ranges can be used to achieve substantially the same results as values within the ranges. Also, unless indicated otherwise, the disclosure of these ranges is intended as a continuous range including every value between the minimum and maximum values. For definitions provided herein, those definitions refer to word forms, cognates and grammatical variants of those words or phrases. For definitions provided herein, those definitions also refer to word forms, cognates and grammatical variants of those words or phrases. Also, it should be understood that any numerical range recited herein is intended to include all sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value of equal to or less than 10. The articles “a” and “an” are used herein to refer to one or to more than one (i.e., to at least one) of the grammatical object of the article. By way of example, “an element” means one element or more than one element.

Described herein are methods of making a CNS ECM preparation suitable for use in tissue engineering and regenerative medicine applications, such as replacement of CNS tissue along with a CNS ECM preparation prepared by those methods and methods of using the CNS ECM preparations. In certain non-limiting embodiments, the CNS ECM preparation comprises decellularized spinal cord tissue or brain tissue. The CNS ECM material may be a solution, gel or powder. In another non-limiting embodiment, the CNS ECM preparations comprise bioactive or therapeutic agents. Of note, the methods described herein for preparation of CNS ECM are substantially different from methods traditionally used to prepare ECM materials, such as decellularized UBM and SIS ECM preparations as described below, which methods are ineffective to produce a useful decelluarized CAN-ECM product.

ECM preparations can be used for a large number of medical applications including, but not limited to, wound healing, tissue remodeling, and tissue regeneration. For example and without limitation, such ECM preparations can be used for wound healing. In one non-limiting embodiment, the ECM preparation comprises bioactive agents to facilitate tissue healing, tissue remodeling and/or angiogenesis. In another non-limiting embodiment, the ECM preparation comprises bioactive agents to ward off bacteria and other pathogens. In yet another non-limiting embodiment, the ECM preparation comprises pores to allow a wound to drain. In yet another non-limiting embodiment, the ECM preparation comprises combinations of cells and bioactive agents. In yet another non-limiting embodiment, combinations of cells and bioactive agents are added to the ECM preparation before or during implantation at a site in a patient.

As used herein, the term “polymer” refers to both synthetic polymeric components and biological polymeric components. The CNS ECM preparations may only consist of CNS-derived ECM material or may comprise CNS ECM material and one or more additional synthetic and/or biological polymeric components. “Biological polymer(s)” (e.g., biological polymeric components) are polymer compositions that are obtained from biological sources, such as, without limitation, mammalian or vertebrate tissue or cells, as in the case of certain ECM compositions, such as UBM and SIS ECM preparations or collagen or cell culture. Biological polymers can be modified by additional processing steps. Polymer(s), in general include, for example and without limitation, mono-polymer(s), copolymer(s), polymeric blend(s), block polymer(s), block copolymer(s), cross-linked polymer(s), non-cross-linked polymer(s), linear-, branched-, comb-, star-, and/or dendrite-shaped polymer(s), where polymer(s) can be formed into any useful form, for example and without limitation, a hydrogel.

Generally, the polymeric components suitable for the CNS ECM preparation described herein may be any polymer that non-cytotoxic and which may be biodegradable.

By “biodegradable”, it is meant that a polymer, once implanted and placed in contact with bodily fluids and/or tissues, will degrade either partially or completely through chemical, biochemical and/or enzymatic processes. Non-limiting examples of such chemical reactions include acid/base reactions, hydrolysis reactions, and enzymatic cleavage.

By “biocompatible,” it is meant that a polymer composition and its normal degradation in vivo products are cytocompatible and are substantially non-toxic and non-carcinogenic in a patient within useful, practical and/or acceptable tolerances. By “cytocompatible,” it is meant that the polymer can sustain a population of cells and/or the polymer composition, device, and degradation products, thereof are not cytotoxic and/or carcinogenic within useful, practical and/or acceptable tolerances. For example, the polymer when placed in a human cell culture does not adversely affect the viability, growth, adhesion, motility, and number of cells. In one non-limiting embodiment, the compositions, and/or devices are “biocompatible” to the extent they are acceptable for use in a human patient according to applicable regulatory standards in a given jurisdiction. In another example the biocompatible polymer, when implanted in a typical patient, does not cause a substantial adverse reaction or substantial harm to cells and tissues in the body, for instance, the polymer composition or device does not cause necrosis or an infection resulting in harm to tissues from the implanted materials.

According to one embodiment, the method for manufacturing (e.g., making or preparing) a CNS ECM preparation comprises freezing CNS tissue, such as spinal cord or brain tissue, and thawing the CNS tissue, typically while agitating the tissue in water, or an equivalent solution, such as a salt solution and/or a buffer solution, including, for example, normal saline (0.9% NaCl), phosphate-buffered saline, Tris, or Tris-EDTA solutions.

In most cases, excised CNS tissue is ensheathed in the dura mater membrane. The membrane can be included in the CNS ECM preparation, but typically is removed prior to freezing the tissue, or after thawing. Freezing the tissue may be conducted rapidly in liquid nitrogen or dry ice, or placed in a freezer, and the frozen tissue may be stored substantially indefinitely prior to thawing. Typically the sample is frozen well below 0° C. to rapidly and completely freeze the sample, and all constituents thereof. As a non-limiting example, the tissue is frozen in liquid nitrogen and is stored in either liquid nitrogen or in a −80° C. freezer. In another example, the tissue is frozen and stored in a −80° C. freezer. As is readily apparent, both the freezing and storage of the frozen tissue may be accomplished at any temperature below 0° C., and preferably below −20° C., independent of the methods of freezing and the freezing and storage device (e.g., freezer).

One or more of the steps of thawing, digesting, incubating the tissue in a solution comprising a detergent, osmotically shocking and incubating the tissue in a solution comprising an emulsifier may be performed while agitating the sample, and in most cases agitation is preferred. As used herein, “agitating” and “agitation” refer to shaking, rocking or otherwise moving a sample at a frequency (e.g., RPM in an orbital shaker, or Hz (cycles per second) in a reciprocating shaker) or strength that is useful to accomplish a stated task without disrupting ECM integrity to the point that the final ECM product is not useful for its intended purpose. For example and without limitation, agitation is sufficient to disrupt ultrastructure of the tissue over the steps of preparing the ECM material while not being so vigorous so as to completely disrupt ECM integrity to the point that the final ECM product is not useful for its intended purpose. In the process step in which the tissue is enzymatically digested while agitating the tissue, the agitation is not too strong to substantially inhibit the enzyme action on the tissue. Examples of useful rates (frequencies) of agitation include (for example, in reference to an orbital or rotary shaker), 60-250 RPM, for example from 100-250 RPM or from 120-200 RPM, inclusive of frequencies therebetween. In another example, agitation for each step of the process requiring agitation is at a frequency or that accomplishes the stated goal of each step and/or results in the preparation of useful ECM product as described herein. Based on the teachings presented herein, those of ordinary skill can empirically determine useful and optimal agitation frequencies for each step.

At this stage, the tissue is strained (filtered) to remove liquid. Straining is done on a screen, filter, etc. of sufficient pore size to retain the tissue and remove excess liquid. Typically the straining is not performed under pressure—that is, the tissue is not pressed or squeezed—but that is an option. Other methods, such as centrifugation and decanting of liquid, or spin filters, can be used to effectively remove excess liquid, so long as the method does not result in substantial loss of functionality of the end-product.

Between any stages, the tissue can be washed (rinsed, etc.) in water or any useful solution, including without limitation: normal saline (0.9% NaCl), phosphate-buffered saline, Tris, or Tris-EDTA solutions. The volumes and nature of the wash (e.g., rinsing, agitating, filtering, decanting, etc.) is not as critical as the substantial retention of functionality of the end-product of the process.

Next, the tissue is digested with one or more proteases, in a suitable incubation solution. The tissue is typically agitated during the incubation. Other proteases or combinations thereof are expected to be use in the methods described herein, including endopeptidases, exopeptidases, serine proteases, threonine proteases, cysteine proteases, aspartate proteases, metalloproteases, glutamic acid proteases, acid proteases, neutral proteases and basic (alkaline) proteases. Non-limiting examples of proteases include: pepsin, trypsin, chymotrypsin, papain, elastase, collagenase and chondroitinase. Each protease has useful and optimal incubation conditions, including salt concentration, presence of cofactors, temperature, agitation strength, requirement for a chelating agent such as EDTA, and buffer requirements. Proteases that can be used in the same incubation solution may be combined, for example, two proteases having different target amino acids, but overlapping incubation conditions may be combined in the same reaction mixture., a protease, to degrade native tissue architecture before the decellularization process begins. Those of skill in the art recognize that incubation times and temperatures can be varied depending on the amount of tissue. As an example, the tissue is digested using trypsin in, for example, an EDTA-containing solution. In this example, the tissue is digested for 1 hour at 37° C. with agitation in an orbital shaker at 120 rpm.

In a next step, the tissue is incubated and agitated in a solution comprising a first surfactant, such as a detergent, including ionic or non-ionic detergents. Nonionic detergents include as a class detergents having uncharged, zwitterionic hydrophilic headgroups. Non-limiting examples of non-ionic detergents include: Tween, Triton, Brij, and CHAPS (3-[(3-cholamidopropyl)dimethylammonio]-1-propanesulfonate) families of detergents. Examples of specific detergents include: TRITON X-100™ (4-octylphenol polyethoxylate), n-dodecylmaltoside, NONIDET P40™, n-octylglucoside, TWEEN 20. Non-ionic detergents are capable of lysing cells and solubilizing the cell membrane as well as many of the cellular components. In one non-limiting example, TRITON X-100™ is used. For example, in one embodiment the tissue can be placed in sequentially increasing amounts of TRITON X-100™ solutions. In one embodiment, the tissue is incubated in a 3% TRITON X-100™ for a period of about an hour. In certain embodiments, the incubation is performed at 37° C. Agitation or shaking may be performed during the incubation step in an orbital shaker, for example at between 100-250 rpm, for one hour.

Next, the tissue may be subjected to osmotic shock, first by exposure to a hypertonic solution, followed by exposure to a hypotonic solution. “hypotonic” and “hypertonic” are relative terms e.g, in relation to physiological osmolality, but can diverge from this so long as the ultimate goal of osmotic shock is achieved. For instance the hypotonic solution may have a lower osmolality with reference to the hypertonic solution, but still may be hypertonic with respect to physiological osmolality. Nevertheless, physiological hypotonicity is expected to be most effective, with water being a particularly useful, and inexpensive hypotonic solution. The sample is preferably agitated during this step. As a non-limiting example, the tissue is first incubated in hypertonic solution, e.g. 1M sucrose. The hypertonic solution is removed by straining, filtering, centrifugation, etc., and the sample is typically washed in a hypotonic solution to remove residual hypertonic solution. The tissue is then incubated in a hypotonic solution (e.g. deionized water). As above, the tissue is preferably agitated, for example in an orbital shaker at about between 100-250 rpm.

The next step in processing the tissue to a CNS ECM product is to incubate the tissue in a second surfactant that is typically an emulsifier. As an example of suitable treatment is to incubate the tissue in 4% deoxycholate while shaking at about between 100-250 rpm. Other surfactants/emulsifiers can be used in other embodiments. Non-limiting examples of suitable emulsifiers include: lecithin, lecithin-deoxycholate, emulsifying wax, cetearyl alcohol, polysorbate 20, and ceteareth 20, among others. The tissue may then be washed, for example using deionized water for 15 minutes at room temperature while shaken at about between 100-250 rpm, depending on the source of the CNS tissue being processed.

The CNS ECM preparation (product) is typically sterilized. It may be sterilized by any of a number of standard methods without loss of function and to an extent acceptable in common medical or veterinary practice. For example and without limitation, the material can be sterilized by propylene oxide or ethylene oxide treatment, UV radiation, alcohol (e.g., ethanol) sterilization, gamma irradiation treatment (0.05 to 4 mRad), gas plasma sterilization, peracetic acid sterilization, or electron beam treatment. Treatment with glutaraldehyde results in sterilization as well as increased cross-linking of the ECM. Glutaraldehyde and other chemical crosslinking treatment substantially alters the material such that it is slowly resorbed or not resorbed at all and incites a different type of host remodeling, which more closely resembles scar tissue formation or encapsulation rather than constructive remodeling. If desired, cross-linking of the protein material within the ECM can also be induced with, for example and without limitation, carbodiimide isocyanate treatments, dehydrothermal methods, and photooxidation methods.

In one non-limiting embodiment, the ECM is disinfected by immersion in 0.1% (v/v) peracetic acid, 4% (v/v) ethanol, and 96% (v/v) sterile water for two hours. The ECM material is then washed twice for 15 minutes with PBS (pH=7.4) and twice for 15 minutes with deionized water. The ECM-derived material may be further processed by optional drying, desiccation, lyophilization, freeze drying, and/or glassification. The material may be comminuted by any means, for example in a classification mill. The ECM-derived material optionally can be further processed, for example and without limitation by hydration (if dried), acidification, conjugation with another chemical composition, enzymatic digests with, for example and without limitation, trypsin or pepsin, etc. The material may be re-hydrated in any suitable solution, including without limitation, PBS, saline, water and cell culture media.

In certain non-limiting embodiments, the CNS ECM preparation further comprises synthetic or additional biological polymers. Synthetic (co)polymers, may comprise homopolymers, copolymers, and/or polymeric blends comprising, without limitation, one or more of the following monomers: glycolide, lactide, caprolactone, dioxanone, and trimethylene carbonate. In non-limiting embodiments, the synthetic polymer(s) comprise labile chemical moieties, non-limiting examples of which include esters, anhydrides, polyanhydrides, or amides, which can be useful in, for example and without limitation, controlling the degradation rate of the CNS ECM preparation and/or the release rate of therapeutic agents from the CNS ECM preparation. Alternatively, the polymer(s) may contain peptides or biomacromolecules as building blocks which are susceptible to chemical reactions once placed in situ. In one non-limiting example, the polymer is a polypeptide comprising the amino acid sequence alanine-alanine-lysine, which confers enzymatic lability to the polymer. In another non-limiting embodiment, the CNS ECM preparation comprises a biomacromolecular component derived from an ECM other than a CNS-derived ECM material. For example, the polymer composition may comprise the biomacromolecule collagen so that collagenase, which is present in situ, can degrade the collagen.

According to one embodiment, any polymer component(s) added to the CNS ECM preparation are selected so that they degrade in situ on a timescale that is similar to an expected rate of healing of the wound or tissue. Non-limiting examples of in situ degradation rates include between one day and one year or increments therebetween for instance, between two weeks and 10 months, and between 3 to 4 months.

The mechanical properties of the CNS ECM preparation can be optimized by changing the protein concentration of the gel or by adding additional polymeric components to operate under the normal strain and stress on the native tissue at the site of implantation. In certain non-limiting embodiments, the mechanical properties of the CNS ECM preparation are optimized similar to or identical to that of native tissue. In one non-limiting embodiment, the CNS ECM preparation is injected in liquid or gel form into a predetermined location, e.g. the site of injury, such as in or about the spinal cord, within the patient.

In many instances, the gel is a reverse-phase gel where the solution is a liquid at a first temperature and a gel at a second, higher temperature. For example, a gel may be a liquid below (e.g.) 20° C., 25° C. or 30° C., but a gel at 37° C. such that when room-temperature material, in liquid form, is injected into a patient, the material gels. In one embodiment, the CNS ECM preparation is a liquid with a protein concentration exceeding 2.0 mg/ml and is injected at the site of interest, where it forms a gel. In another embodiment, the CNS ECM preparation is a liquid which is combined with a non-CNS ECM preparation. Their combined protein concentration exceeds 2.0 mg/ml and they are mixed thoroughly and injected at a site of interest, where they form a combination CNS and non-CNS gel. Both brain ECM and spinal cord ECM digests prepared according to the methods provided herein are liquid at room temperature and then can be gelled at 37° C. For the brain ECM digest, it is desirable to first produce a particulate that is less than 840 microns to facilitate gelation. In particular illustrative experiments, the storage moduli for spinal cord ECM digest ranged from 190 to 320 with an average of 240 Pa (n=3) and the storage moduli for brain ECM ranges from 40 to 70 Pa with an average of 56 Pa (n=2).

The mechanical properties of the CNS ECM preparation also may be optimized to be suitable for surgical handling. In certain embodiments the CNS ECM preparation is a gel and has gel like properties that can be controlled by the degree of hydration. For example, the gel can be a hydrogel and be semi-solid, thus having a three dimensional structure. In another non-limiting embodiment, the CNS ECM preparation is a flexible sheet and can be sutured to the site. In another, the CNS ECM preparation is foldable and can be delivered to the site by minimally invasive laparoscopic methods.

The additional polymer components added to the CNS ECM preparation also can be elastomeric. Generally, an elastomeric polymer that has properties similar to that of the soft tissue to be replaced or repaired is appropriate. For example, in certain embodiments, the polymers used to make a device is distensible. Non-limiting examples of suitable polymers include those that have a breaking strain of from 100% to 1700%, more preferably between 200% and 800%, and even more preferably between 325% and 600%. In particularly preferred embodiments, the breaking strain of the polymer is between 5% and 50%, more preferably between 10% and 40%, and even more preferably between 20% and 30%. Further, it is often useful to select polymers with tensile strengths of from 10 kPa-30 MPa, more preferably from 5-25 MPa, and even more preferably between 8 and 20 MPa. In certain embodiments, the initial modulus is between 10 kPa to 100 MPa, more preferably between 10 and 90 MPa, and even more preferably between 20 and 70 MPa.

A number of biocompatible, biodegradable elastomeric (co)polymers are known and have been established as useful in preparing cell growth matrices/biocompatible scaffolds, including biodegradable poly(ester urethane) urea (PEUU), poly(ether ester urethane)urea (PEEUU), poly(ester carbonate)urethane urea (PECUU) and poly(carbonate)urethane urea (PCUU). In general, useful copolymers comprise monomers derived from alpha-hydroxy acids including polylactide, poly(lactide-co-glycolide), poly(L-lactide-co-caprolactone), polyglycolic acid, poly(dl-lactide-co-glycolide), poly(l-lactide-co-dl-lactide); monomers derived from esters including polyhydroxybutyrate, polyhydroxyvalerate, polydioxanone and polygalactin; monomers derived from lactones including polycaprolactone; monomers derived from carbonates including polycarbonate, polyglyconate, poly(glycolide-co-trimethylene carbonate), poly(glycolide-co-trimethylene carbonate-co-dioxanone); monomers joined through urethane linkages, including polyurethane, poly(ester urethane) urea elastomer.

In one example, as described in US Patent Publication No. 20080268019, incorporated herein by reference for its technical disclosure, the polymer composition comprises a synthetic polymeric component and a biological polymeric component. The synthetic polymeric component may comprise a thermoplastic biodegradable elastomer, and/or the biological polymeric component may comprise an extracellular matrix-derived material, such as a CNS ECM-derived material as described herein. The synthetic polymeric component may comprise one or more of a PEUU, PEEUU, PECUU or PCUU elastomer. The elastomer may comprise a diamine, such as putrescine or lysine ethyl ester, or a diol. The elastomer may comprise a polycaprolactone or a polycaprolactone diol, such as a triblock copolymer comprising polycaprolactone or a polycaprolactone-b-polyethylene glycol-b-polycaprolactone triblock copolymer. The elastomer can be functionalized with an adhesion-promoting peptide, such as the oligopeptide RGD. In one non-limiting embodiment, the elastomer comprises an isocyanate derivative, a polycaprolactone diol, and a diamine chain extender, which may comprise a ratio of isocyanate derivative:polycaprolactone diol:diamine chain extender of about 2:1:1. In another non-limiting embodiment, the elastomer comprises an isocyanate derivative, a triblock copolymer comprising polycaprolactone, and a diamine chain extender in which the ratio of isocyanate derivative:triblock copolymer:diamine chain extender optionally is about 2:1:1. For example, in one non-limiting embodiment the molar feed ratio for preparation of PEUU is: polycaprolactone:diisocyanatobutane:putrescine=1:2:1, and the molar feed ratio for synthesis of PEEUU is:

polycaprolactone-PEG-polycaprolactone copolymer:diisocyanatobutane:putrescine=1:2:1.

Other useful copolymers include: polylactide, polyglycolide, poly(lactide-co-glycolide), polycaprolactone, poly(lactide-co-caprolactone), poly(trimethylene carbonate) based polymers, polyhydroxybutyrate and its copolymer, polydioxanone, poly(ester carbonate urethane) urea, poly(carbonate urethane) urea, polycarbonate urethane, or polyester urethane. In another embodiment, natural polymers are included in the polymer composition, including gelatin, collagen, chitosan, hyaluronic acid, etc.

A poly(ester urethane) urea elastomer (PEUU) may be made from polycaprolactonediol (MW 2,000) and 1,4-diisocyanatobutane, with a diamine, such as putrescine as the chain extender. A suitable PEUU polymer may be made by a two-step polymerization process whereby polycaprolactone diol (Mw 2,000), 1,4-diisocyanatobutane, and putrescine are combined in a 1:2:1 molar ratio though virually any molar feed ratio may suffice so long as the molar ratio of each monomer component is >0. In one embodiment, the molar feed ratio of polycaprolactone diol plus putrescine is equal to that of diisocyanatobutane. In the first polymerization step, a 15 wt % solution of 1,4-diisocyanatobutane in DMSO is stirred continuously with a 25 wt % solution of diol in DMSO. In the second step, stannous octoate is added and the mixture is allowed to react at 75° C. for 3 hours, with the addition of triethylamine to aid dissolution. A poly(ether ester urethane) urea elastomer (PEEUU) may be made by reacting polycaprolactone-b-polyethylene glycol-b-polycaprolactone triblock copolymers with 1,4-diisocyanatobutane and putrescine. In a preferred embodiment, PEEUU is obtained by a two-step reaction using a 2:1:1 reactant stoichiometry of 1,4-diisocyanatobutane:triblock copolymer:putrescine. In the first polymerization step, a 15 wt % solution of 1,4-diisocyanatobutane in DMSO is stirred continuously with a 25 wt % solution of triblock compolymer diol in DMSO. In the second step, stannous octoate is added and the mixture is allowed to react at 75° C. for 3 hours. The reaction mixture is then cooled to room temperature and allowed to continue for 18 h. The PEEUU polymer solution is then precipitated with distilled water and the wet polymer is immersed in isopropanol for 3 days to remove unreacted monomer and dried under vacuum.

Poly(ester carbonate)urethane urea (PECUU) and poly(carbonate)urethane urea (PCUU) are described, for example, in Hong at al. (Tailoring the degradation kinetics of poly(ester carbonate urethane)urea thermoplastic elastomers for tissue engineering scaffolds Biomaterials, doi:10.1016/j.biomaterials.2010.02.005). Poly(ester carbonate urethane)urea (PECUU) is synthesized, for example using a blended soft segment of polycaprolactone (PCL) and poly(1,6-hexamethylene carbonate) (PHC) and a hard segment of 1,4-diisocyanatobutane (BDI) with chain extension by putrescine. Different molar ratios of PCL and PHC can be used to achieve different physical characteristics. Putrescine is used as a chain extender by a two-step solvent synthesis method. In one example, the (PCL+PHC):BDI:putrescine molar ratio is defined as 1:2:1. Variable molar ratios of PCL and PHC (e.g., PCL/PHC ratios of 100/0 (yielding a PEUU), 75/25, 50/50, 25/75 and 0/100 (yielding a PCUU)) are completely dissolved in DMSO in a 3-neck flask with argon protection and then BDI is added to the solution, following 4 drops of Sn(Oct)₂. The flask is placed in an oil bath at 70° C. After 3 h, the prepolymer solution is cooled at room temperature and then a putrescine/DMSO solution is added dropwise into the agitated solution. The final polymer solution concentration is controlled to be approximately 4% (w/v). Then the flask is than placed in an oil bath and kept at 70° C. overnight. The polymer is precipitated in an excess volume of cool deionized water and then dried in a vacuum at 60° C. for 3 days. The polyurethane ureas synthesized from the different PCL/PHC molar ratios defined above are referred to as PEUU, PECUU 75/25, PECUU 50/50, PECUU 25/75 and PCUU, respectively. In practice, the yields of all final products using this method is approximately 95%.

According to one non-limiting embodiment, the additional polymer component comprises one or both of a collagen and an elastin. Collagen is a common ECM component and typically is degraded in vivo at a rate faster than many synthetic bioerodable polymers. Therefore, manipulation of collagen content in the CNS ECM preparation can be used as a method of modifying bierosion rates in vivo. Collagen may be present in the polymer composition in any useful range, including, without limitation, from about 2% wt. to about 95% wt., but more typically in the range of from about 25% wt. to about 75% wt., inclusive of all ranges and points therebetween, including from about 40% wt. to about 75% wt., including about 75% wt. and about 40% wt. Elastin may be incorporated into the polymer composition in order to provide increased elasticity. Elastin may be present in the polymer composition in any useful range, including without limitation, from about 2% wt. to about 50% wt., inclusive of all ranges and points therebetween, including about 40% wt., inclusive of all integers and all points therebetween and equivalents thereof. In one non-limiting embodiment, collagen and elastin are present in approximately equal amounts in the polymer composition, In another embodiment, the sum of the collagen and elastin content in the polymer composition is in any useful range, including, without limitation, from about 2% wt. to about 95% wt., but more typically in the range of from about 25% wt. to about 75% wt., inclusive of all ranges and points therebetween, including from about 40% wt. to about 75% wt., including about 75% wt. and about 40% wt.

Generally, any type of extracellular matrix (ECM) can be used to prepare the additional ECM material (biological polymeric component) of the CNS ECM preparation (for example and without limitation, see U.S. Pat. Nos. 4,902,508; 4,956,178; 5,281,422; 5,352,463; 5,372,821; 5,554,389; 5,573,784; 5,645,860; 5,771,969; 5,753,267; 5,762,966; 5,866,414; 6,099,567; 6,485,723; 6,576,265; 6,579,538; 6,696,270; 6,783,776; 6,793,939; 6,849,273; 6,852,339; 6,861,074; 6,887,495; 6,890,562; 6,890,563; 6,890,564; and 6,893,666; each of which is incorporated by reference in its entirety for its technical disclosure). As used herein, the terms “extracellular matrix” and “ECM” refer to a complex mixture of structural and functional biomolecules and/or biomacromolecules including, but not limited to, structural proteins, specialized proteins, proteoglycans, glycosaminoglycans, and growth factors that surround and support cells within mammalian tissues. By “ECM material” it is meant a composition that is prepared from a natural tissue and ECM or from an in vitro source wherein the ECM is produced by cultured cells and comprises polymeric components (constituents) of native ECM. Additionally, “decellularized” ECM refers to ECM in which the cells have been removed through processes described herein and known in the art. Of note, “ECM material” or ECM-derived material” does not include as a class pure or substantially pure preparations of specific ECM proteins or peptides that are isolated and purified from ECM material, resulting in an essentially or substantially pure, homogenous preparation of one protein or peptide from the ECM, such as essentially pure preparations of collagen, elastin, heparin sulfate, chondroitin sulfate, keratin sulfate, hyaluronic acid, fibronectin, laminin or growth factors, as are commercially available.

According to one non-limiting example of the additional ECM material, ECM is isolated from a vertebrate animal, for example, from a warm blooded mammalian vertebrate animal including, but not limited to, human, monkey, pig, cow, sheep, etc. The ECM may be derived from any organ or tissue, including without limitation, urinary bladder, intestine, liver, heart, esophagus, spleen, stomach and dermis. The ECM can comprise any portion or tissue obtained from an organ, including, for example and without limitation, submucosa, epithelial basement membrane, tunica propria, etc. In one non-limiting embodiment, the ECM is isolated from urinary bladder, which may or may not include the basement membrane. In another non-limiting embodiment, the ECM includes at least a portion of the basement membrane. In certain non-limiting embodiments, the CNS ECM preparation comprises from 1% to 75% of the additional ECM material. The ECM material may or may not retain some of the cellular elements that comprised the original tissue such as capillary endothelial cells or fibrocytes. The type of additional ECM used in the CNS ECM preparation can vary depending on the intended cell types to be recruited during wound healing or tissue regeneration, the native tissue architecture of the tissue organ to be replaced, the availability of the tissue source of ECM, or other factors that affect the quality of the final CNS ECM preparation and the possibility of manufacturing the CNS ECM preparation. For example and without limitation, the additional ECM may contain both a basement membrane surface and a non-basement membrane surface, which would be useful for promoting the reconstruction of tissue such as the urinary bladder, esophagus, or blood vessel all of which have a basement membrane and non-basement membrane component.

Commercially available ECM preparations can also be used as the additional biological polymeric component of the CNS ECM preparation. In one non-limiting embodiment, the additional ECM is derived from small intestinal submucosa or SIS. Commercially available preparations include, but are not limited to, SURGISIS™′ SURGISIS-ES™, STRATASIS™, and STRATASIS-ES™ (Cook Urological Inc.; Indianapolis, Ind.) and GRAFTPATCH™ (Organogenesis Inc.; Canton, Mass.). In another non-limiting embodiment, the ECM is derived from dermis. Commercially available preparations include, but are not limited to PELVICOL™ (sold as PERMACOL™ in Europe; Bard, Covington, Ga.), REPLIFORM™ (Microvasive; Boston, Mass.) and ALLODERM™ (LifeCell; Branchburg, N.J.). In another embodiment, the additional ECM is derived from urinary bladder. Commercially available preparations include, but are not limited to UBM (Acell Corporation; Jessup, Md.).

The CNS ECM preparation may take many different forms. As indicated herein, according to one embodiment, the material is a gel or a powder. Where the CNS ECM preparation is sufficiently concentrated (high % wt.), or when combined with additional biological or synthetic polymer components, it will exhibit structural integrity and can be molded or otherwise fabricated into a variety of shapes. In certain non-limiting embodiments, the CNS ECM preparation is formed as a woven or nonwoven flexible fabric that can be sewn directly on to the site to be treated. The CNS ECM preparation can be affixed in place at the site of implantation or affixed using a medically acceptable adhesive. In one non-limiting embodiment, the CNS ECM preparation is substantially planar (having much greater dimension in two dimensions and a substantially smaller dimension in a third, comparable to bandages, gauze, and other substantially flexible, flat items). In another non-limiting embodiment, the CNS ECM preparation is (either solid or hollow) tubular, and can be attached by anastamosis to tissue such as intestine or blood vessel tissue. The matrix can be electrodeposited, for example, with different polymers being deposited at different locations and/or electrosprayed with different media at different times, for instance containing different growth factors, biological components, etc. to generate a 3-dimensional structure that may be used for partial or complete organ replacement.

The CNS ECM preparation can also have or be formed into three-dimensional shapes (that is devices) useful for treating wounds and tissue deficiencies, such as plugs, rings, wires, cylinders, tubes, or disks. A useful range of thickness for the CNS ECM preparation is between from about 10 μm (micrometers or microns (p)) to about 3.5 cm, including increments therebetween, including, without limitation from about 10 μm to about 50 μm, 50 μm to 3.5 cm, 100 μm to 3.0 cm, and between 300 μm and 2.5 cm. The CNS ECM preparation may be molded or otherwise formed into a device, either as a powder or gel, and optionally surrounded by, e.g., a sheet of ECM material, as a pouch or other shape, such as plugs, rings, wires, cylinders, tubes, or disks, for instance as a nerve guide.

In certain non-limiting embodiments, the formation and initial processing of the synthetic polymeric component and the biological polymeric component (e.g., the CNS-derived ECM material and any additional biological materials, such as ECM) are separate. For example, the synthesis and dissolution of the synthetic polymeric component may involve solvents that would adversely affect the desirable biological properties of the biological polymeric component. By performing the synthesis and initial processing of the synthetic polymeric component separately from the corresponding synthesis and initial processing steps of the biological polymeric component, it is possible to substantially protect the biological polymeric component against degradation that it would otherwise face when exposed to the solvents used in the synthesis and processing the synthetic polymeric component. In certain non-limiting embodiments, the synthetic polymeric component and biological polymeric component are dispersed in different solvents and subsequently combined (e.g., by electrospraying the biological component onto the polymer matrix as it is being electrodeposited). For example, the electrosprayed media may comprise dissolved/solubilized collagen, elastin, CNS-derived ECM material, additional ECM material or other biopolymer(s).

Variables that can be optimized include without limitation, the extent of physical cross-linking in a network comprising polymeric components, the ratio of polymeric components within the network, the distribution of molecular weight of the polymeric components, and the method of processing the polymers. Polymers are typically semicrystalline and their physical properties and/or morphology are dependent upon a large number of factors, including monomer composition, polydispersity, average molecular weight, cross-linking, and melting/crystallization conditions. For example, flow and/or shear conditions during cooling of a polymer melt are known to affect formation of crystalline structures in the composition. In one non-limiting embodiment, the CNS-derived ECM material comprises a polymeric component that provides strength and durability to the CNS-derived ECM material, yet is elastomeric so that the mechanical properties of the CNS-derived ECM material are similar to the native tissue surrounding the wound or site in need of tissue regeneration.

The CNS ECM preparation can have an activated surface, which can be modified with an adhesion-promoting oligopeptide to promote cellular ingrowth into and/or onto the CNS ECM preparation. Non-limiting examples of adhesion-promoting oligopeptides include: RGD or RGDS (SEQ ID NO.: 1), a recognition site for fibronectin, vitronectin, fibrinogen, von Willebrand factor, and collagen; LDV, REDV (SEQ ID NO.: 2), PHSRN (SEQ ID NO.: 3), and KNEED (SEQ ID NO.: 4), which are recognition sites for fibronectin; YIGSR (SEQ ID NO.: 5) and IKVAV (SEQ ID NO.: 6), which are recognition sites for laminin; and DGEA (SEQ ID NO.: 7), a recognition site for collagen.

One or more of therapeutic agents can be introduced into the CNS ECM preparation by any useful method, such as, without limitation absorption, adsorption, deposition, admixture with a polymer composition used to manufacture the CNS ECM preparation and linkage of the agent to a component of the CNS ECM preparation. In one non-limiting example, the therapeutic agent is introduced into a backbone of a polymer composition used in the CNS ECM preparation. By adding the therapeutic agent to the polymer composition itself, the rate of release of the therapeutic agent may be controlled by the rate of polymer degradation. In another non-limiting example, the therapeutic agent is introduced when the CNS ECM preparation is being made. For instance, during hydration of a dehydrated (e.g., lyophilized) CNS ECM preparation, the therapeutic agent can be added to the mixture comprising the CNS ECM preparation, either before (e.g., as a dry powder), during or after hydration. During an electrodeposition process, the therapeutic agent can be electrosprayed onto the polymer being spun. In yet another non-limiting example, the therapeutic agent is introduced into the CNS ECM preparation after a solid gel structure is formed is made. For instance, the CNS ECM preparation may be “loaded” with therapeutic agent(s) by using static methods. For instance, the CNS ECM preparation can be immersed into a solution containing the therapeutic agent, permitting the agent to absorb into and/or adsorb onto the CNS ECM preparation. The CNS ECM preparation may also be loaded by using dynamic methods. For instance, a solution containing the therapeutic agent can be perfused. In another instance, a therapeutic agent can be added to the biodegradable CNS ECM preparation before it is injected/implanted in the patient.

Therapeutic agents within the CNS ECM preparation can be used in any number of ways. In one non-limiting embodiment, a therapeutic agent is released from the CNS ECM preparation. For example and without limitation, anti-inflammatory drugs are released from the CNS ECM preparation to decrease an adverse immune response. In another non-limiting embodiment, a therapeutic agent is intended to substantially remain within the CNS ECM preparation. For example and without limitation, chemoattractants are maintained within the CNS ECM preparation to promote cellular migration and/or cellular infiltration into the CNS ECM preparation.

In one non-limiting embodiment, the CNS ECM preparation release therapeutic agents when the polymeric components degrade within the patient's body. For example and without limitation, the individual building blocks of the polymers may be chosen such that the building blocks themselves provide a therapeutic benefit when released in situ through the degradation process. In one non-limiting embodiment, one of the polymer building blocks is putrescine, which has been implicated as a substance that causes cell growth and cell differentiation.

In another non-limiting embodiment, at least one therapeutic agent is added to the CNS ECM preparation before it is implanted in the patient. Generally, the therapeutic agents include any substance that can be coated on, embedded into, absorbed into, adsorbed onto, or otherwise attached to or incorporated onto or into the CNS ECM preparation that would provide a therapeutic benefit to a patient. Non-limiting examples of such therapeutic agents include antimicrobial agents, growth factors, emollients, retinoids, and topical steroids. Each therapeutic agent may be used alone or in combination with other therapeutic agents. For example and without limitation, a CNS ECM preparation comprising neurotrophic agents or cells that express neurotrophic agents may be applied to a wound that is near a critical region of the central nervous system, such as the spine. Alternatively, the therapeutic agent may be blended with the polymer while the polymer is being processed. For example, the therapeutic agent may be dissolved in a solvent (e.g., DMSO) and added to the polymer blend during processing. In another embodiment, the therapeutic agent is mixed with a carrier polymer (e.g., polylactic-glycolic acid microparticles) which can be added during the processing of the CNS ECM preparation. By blending the therapeutic agent with a carrier polymer, the rate of release of the therapeutic agent may be controlled by the rate of polymer degradation.

While the disclosed CNS ECM preparation demonstrates retention of native growth factors, such as in certain non-limiting embodiments, the therapeutic agent is a growth factor, such as basic fibroblast growth factor (bFGF), vascular endothelial growth factor (VEGF), and transforming growth factors α and β (TGF-α and TGF-β) Additional neurotrophic or angiogenic factors, which optionally may be prepared using recombinant techniques, can be added to the CNS ECM preparation. Non-limiting examples of growth factors include (bFGF), acidic fibroblast growth factor (aFGF), VEGF, hepatocyte growth factor (HGF), insulin-like growth factors 1 and 2 (IGF-1 and IGF-2), platelet derived growth factor (PDGF), stromal derived factor 1 alpha (SDF-1 alpha), nerve growth factor (NGF), ciliary neurotrophic factor (CNTF), neurotrophin-3, neurotrophin-4, neurotrophin-5, pleiotrophin protein (neurite growth-promoting factor 1), midkine protein (neurite growth-promoting factor 2), brain-derived neurotrophic factor (BDNF), tumor angiogenesis factor (TAF), corticotrophin releasing factor (CRF), transforming growth factors α and β (TGF-α and TGF-β), interleukin-8 (IL-8), granulocyte-macrophage colony stimulating factor (GM-CSF), interleukins, and interferons. Commercial preparations of various growth factors, including neurotrophic and angiogenic factors, are available from R & D Systems, Minneapolis, Minn.; Biovision, Inc, Mountain View, Calif.; ProSpec-Tany TechnoGene Ltd., Rehovot, Israel; and CELL SCIENCES®, Canton, Mass.

Methods of promoting wound healing or tissue generation or regeneration in a patient also are provided. The methods comprise, without limitation, implanting/injecting a CNS ECM preparation as described herein at or near a site for wound healing or tissue generation or regeneration in the patient. Although the CNS ECM preparation may be injected or otherwise applied in a patient at any site, the CNS ECM preparation is shown to support neurite growth and therefore can be applied to the CNS, including at a site of spinal cord or brain injury. In any such method, the CNS ECM preparation may comprise a therapeutic agent as described herein.

In certain non-limiting embodiments, the therapeutic agent is an antimicrobial agent, such as, without limitation, isoniazid, ethambutol, pyrazinamide, streptomycin, clofazimine, rifabutin, fluoroquinolones, ofloxacin, sparfloxacin, rifampin, azithromycin, clarithromycin, dapsone, tetracycline, erythromycin, ciprofloxacin, doxycycline, ampicillin, amphotericin B, ketoconazole, fluconazole, pyrimethamine, sulfadiazine, clindamycin, lincomycin, pentamidine, atovaquone, paromomycin, diclazaril, acyclovir, trifluorouridine, foscarnet, penicillin, gentamicin, ganciclovir, iatroconazole, miconazole, Zn-pyrithione, and silver salts such as chloride, bromide, iodide and periodate.

In certain non-limiting embodiments, the therapeutic agent is an anti-inflammatory agent, such as, without limitation, a NSAID, such as salicylic acid, indomethacin, sodium indomethacin trihydrate, salicylamide, naproxen, colchicine, fenoprofen, sulindac, diflunisal, diclofenac, indoprofen, sodium salicylamide; an anti-inflammatory cytokine; an anti-inflammatory protein; a steroidal anti-inflammatory agent; or an anti-clotting agents, such as heparin. Other drugs that may promote wound healing and/or tissue regeneration may also be included.

In certain non-limiting embodiments, the therapeutic agent comprises cells that are added to the CNS ECM preparation before or at the time of implantation/injection. Cells may be autologous (obtained from the patient to receive the CNS ECM preparation), from an allogeneic or xenogeneic source or from any useful cell line, such as, without limitation, stem cells that are capable of cellular growth, remodeling, and/or differentiation. By way of example only, the cells that may be incorporated onto or into the biodegradable CNS ECM preparation include stem cells, precursor cells, smooth muscle cells, skeletal myoblasts, myocardial cells, endothelial cells, and genetically modified cells. Various commercially available cell lines include CLONETICS® Primary Cell Systems (Lonza Group, Inc., Switzerland), ATCC.

In one non-limiting embodiment, the genetically modified cells are capable of expressing a therapeutic substance, such as a growth factor. Cells can be modified by any useful method in the art. For example and without limitation, the therapeutic agent is a growth factor that is released by cells transfected with cDNA encoding for the growth factor. Therapeutic agents that can be released from cells include, without limitation, a neurotrophic factor, such as nerve growth factor, brain-derived neurotrophic factor, neurotrophin-3, neurotrophin-4, neurotrophin-5, and ciliary neurotrophic factor; a growth factor, such as basic fibroblast growth factor (bFGF), acidic fibroblast growth factor (aFGF), vascular endothelial growth factor (VEGF), hepatocyte growth factor (HGF), insulin-like growth factors (IGF), platelet derived growth factor (PDGF), transforming growth factor-beta (TGF-β), pleiotrophin protein (neurite growth-promoting factor 1), and midkine protein (neurite growth-promoting factor 2); an anti-inflammatory cytokine; and an anti-inflammatory protein. The cells may be autologous, allogeneic, etc.

In addition to providing CNS ECM preparation as described above, methods of using such gel CNS ECM preparation are encompassed herein. Generally, a gel CNS ECM preparation can be implanted/injected by using any suitable medical procedure that facilitates use of the CNS ECM preparation to provide a therapeutic benefit.

As used herein, the term “patient” refers to members of the animal kingdom including but not limited to human beings and “mammal” and “mammalian” refers to all warm blooded vertebrates, including, but not limited to human beings. The term “patient” does not imply a doctor-patient relationship. As used herein, the terms “implanted”, “implantation”, “injection” and like terms refer to an act of delivering a CNS ECM preparation to a site within the patient, such as applying the material to the spinal cord or brain (in on or about the spinal cord or brain, for example in, on or about a wound in the spinal cord or brain). The site of implantation in a patient typically is “at or near a site for wound healing or tissue generation or regeneration in the patient,” meaning the CNS ECM preparation-containing device is implanted in, on, onto, adjacent to or in proximity to a desired site of delivery to facilitate healing and/or tissue generation or regeneration to repair an injury or defect in the patient and/or to achieve a desired effect in the patient, such as wound drainage. The delivery method may also include minimally invasive methods such as by catheter based technology or by needle injection. The patient may be human or animal. The CNS ECM preparation may be delivered by any surgical procedure, including minimally invasive techniques, such as laparoscopic surgery, as well as invasive techniques such as neurosurgery.

### Example 1

### Manufacturing Decellularized Spinal Cord Biological Scaffolds

In one example, murine spinal cord was obtained. The spinal cord tissue was transported to the laboratory on ice, wrapped in freezer paper, and stored at −80° C. until needed for ECM derivation processing. The material was thawed and the dura mater was removed from the spinal cord, and the spinal cord was then cut into quarters longitudinally of about 1 inch length and uniform thickness. The spinal cord pieces were placed into water overnight at 4° C. and 120 rpm to mechanically-disrupt the native tissue architecture prior to decellularization. After about 18 h the spinal cord pieces were removed from the water by straining onto a mesh or sieve with hole size of about 840 μm. The pieces of spinal cord were collected with forceps and placed into a flask for protease digestion with 0.02% trypsin/0.05% EDTA solution. The digestion was allowed to proceed in a water bath for 1 h at 37° C. while shaking at 120 rpm. After one hour, the solution was strained off and spinal cord tissue was rinsed gently under a stream of water, detangling as required. The spinal cord pieces were returned to the flask, collecting as many smaller tissue pieces as possible from the strainer using forceps. 3% Triton X-100 solution was then added to the flask to begin decellularization of the tissue, which was placed on a shaker for 1 h at 200 rpm. After one hour the tissue was strained, rinsed, and collected. The tissue pieces were placed back into the flask, and then were subjected to osmotic shock for additional decellularization. Hypertonic 1 M sucrose was added to the flask and placed on a shaker for 15 min at 200 rpm. The tissue was strained, rinsed, collected and combined with hypotonic solution (deionized water) and placed on shaker for 15 min at 200 rpm, to lyse any remaining cells. The decellularized tissue was again strained, rinsed, and reclaimed into a flask. 4% deoxycholate solution was added to the flask and placed upon a shaker for 1 h at 200 rpm. Subsequently, the tissue pieces were strained and rinsed repeatedly in type I (ultrapure) water until all traces of surfactants (bubbles) were removed. The remaining tissue, now enriched into ECM, was collected and disinfected using a peracetic acid solution (made up of Type I water (96%) and 100% EtOH alcohol (4%)) at a ratio of 20:1 peracetic acid solution to weight of ECM, and shaken at 200 rpm for two hours. Following a series of rinse steps in Phosphate Buffered Saline (PBS), the ECM frozen at −20° C. and then lyophilized until all water was removed. Gel scaffolds were manufactured by adding sterile lyophilized ECM into a 0.01 N hydrochloric acid solution containing 1.0 mg/ml pepsin, which is diluted with isotonic saline to yield ECM concentrations between about 1 mg/ml and 200 mg/ml depending upon the desired viscosity of the scaffold.

### Example 2

### Characterization of the Neural-Derived ECM

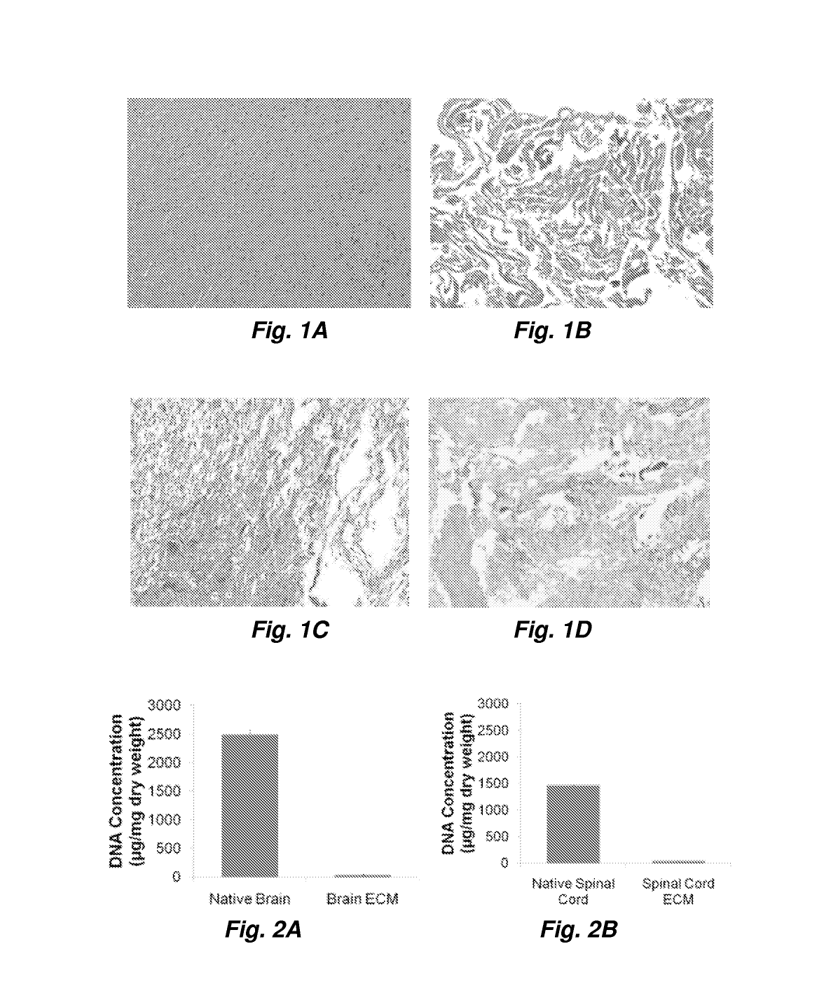

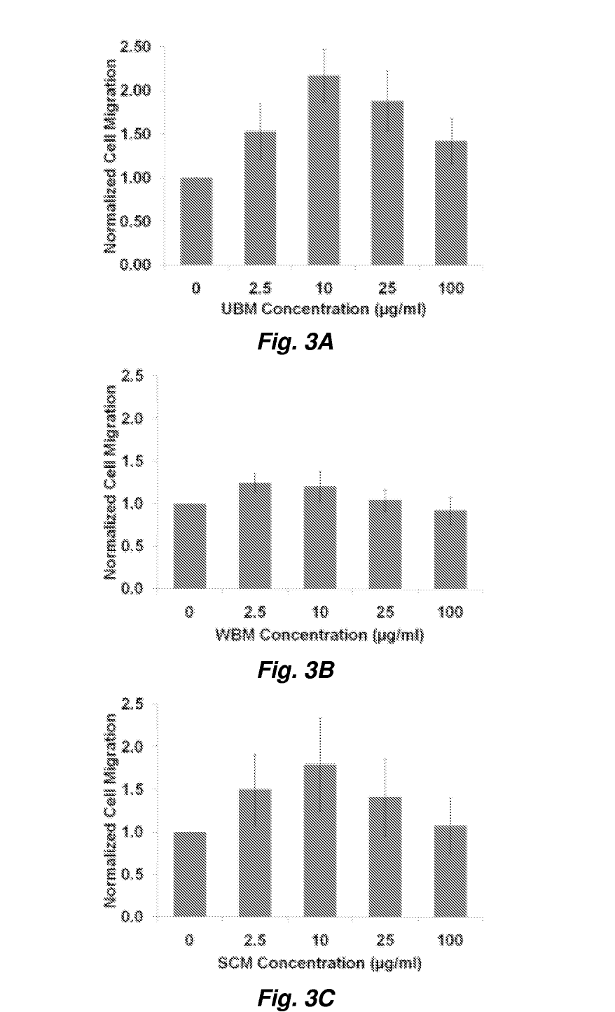

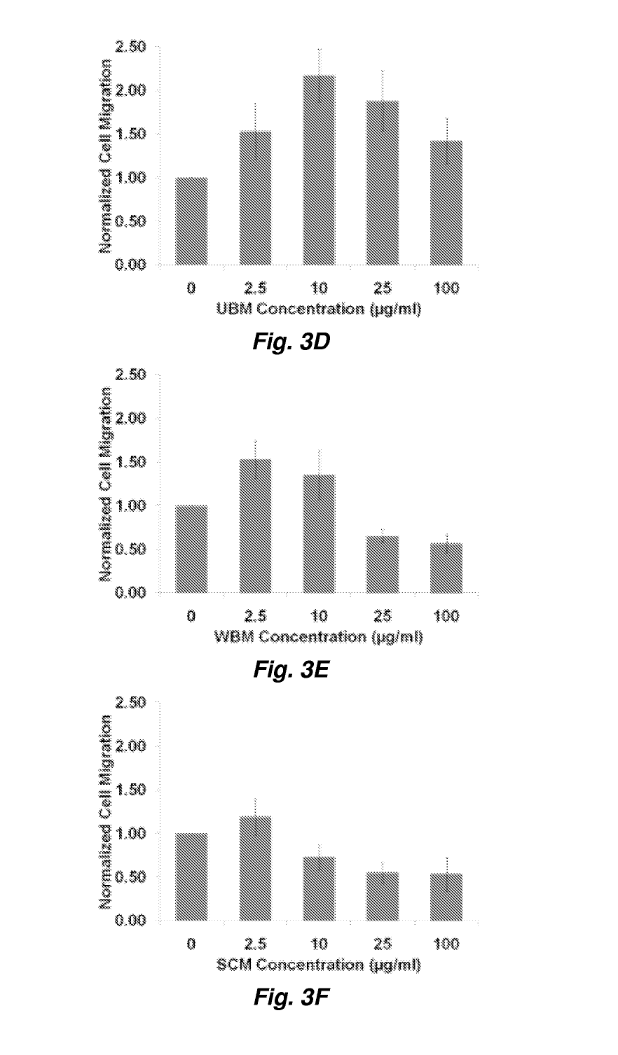

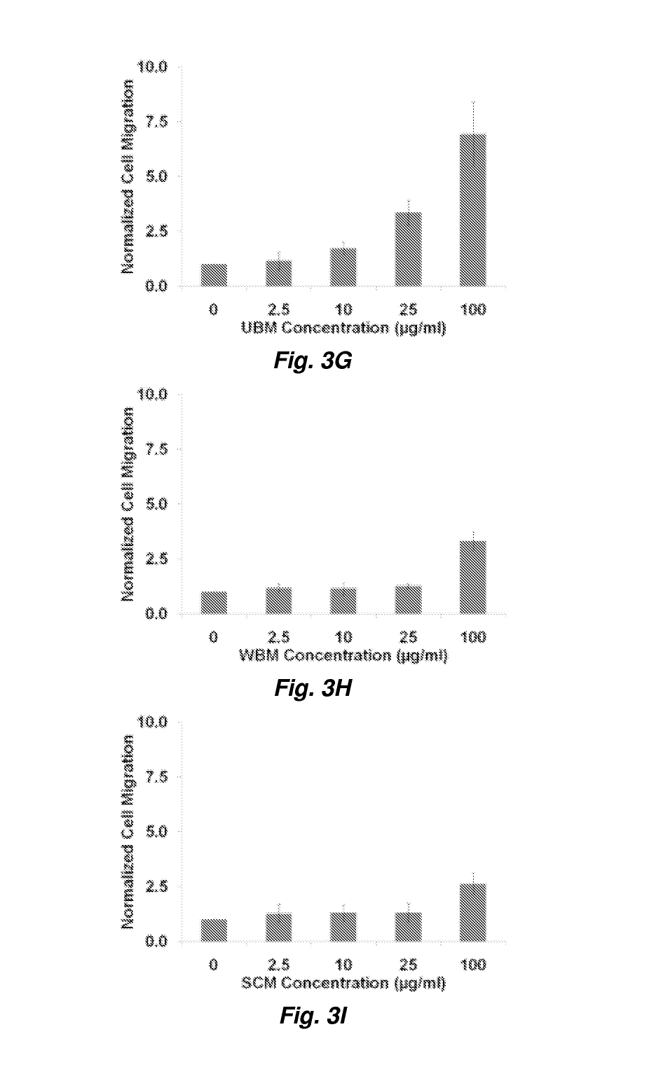

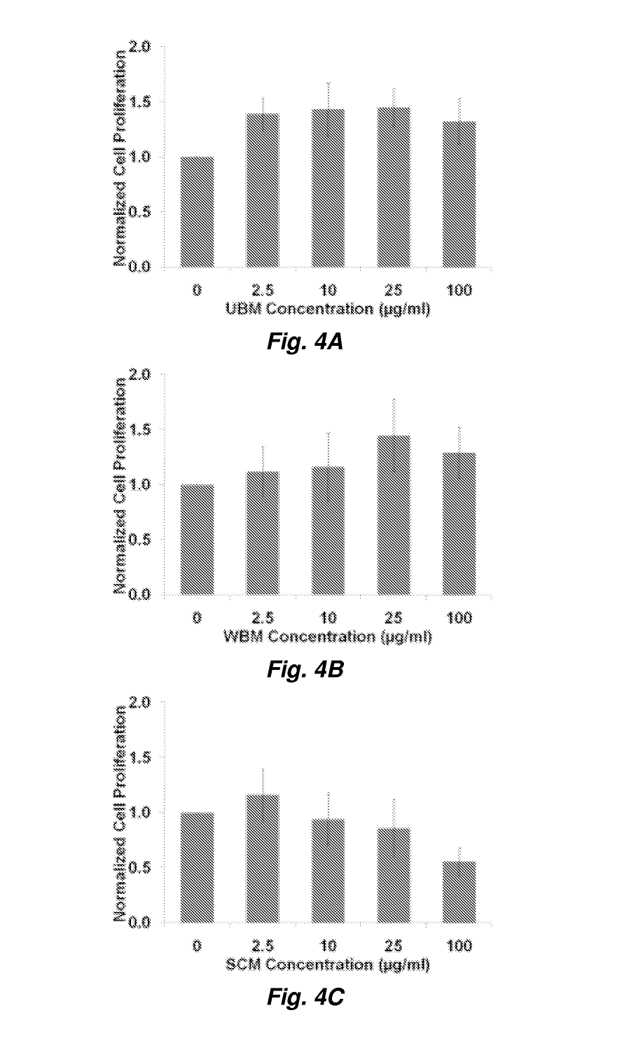

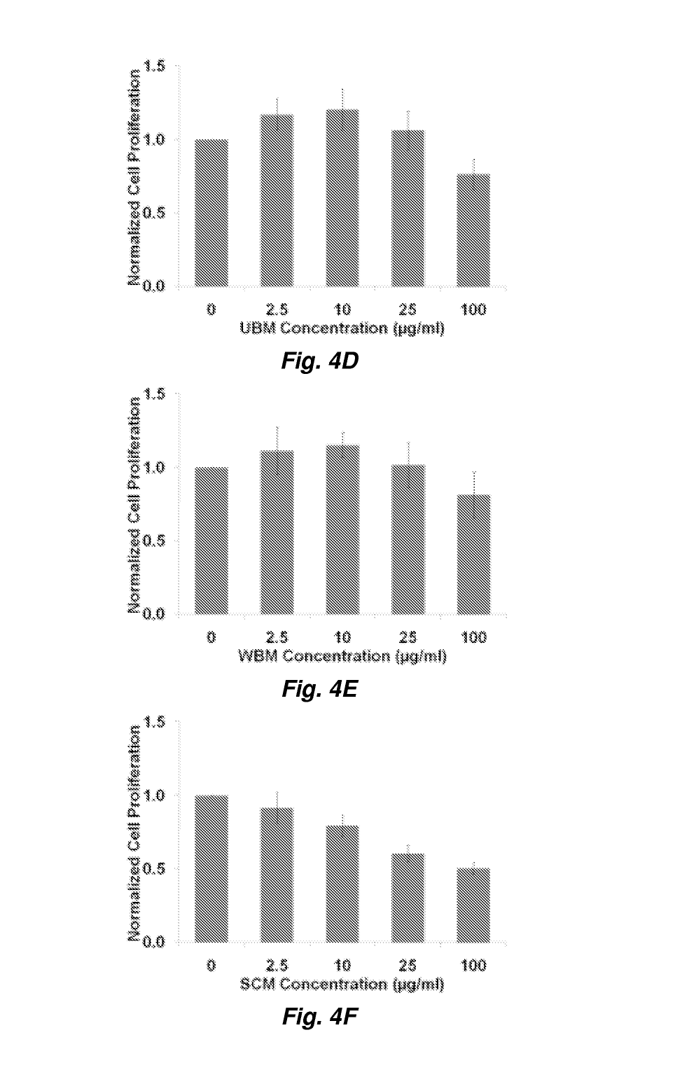

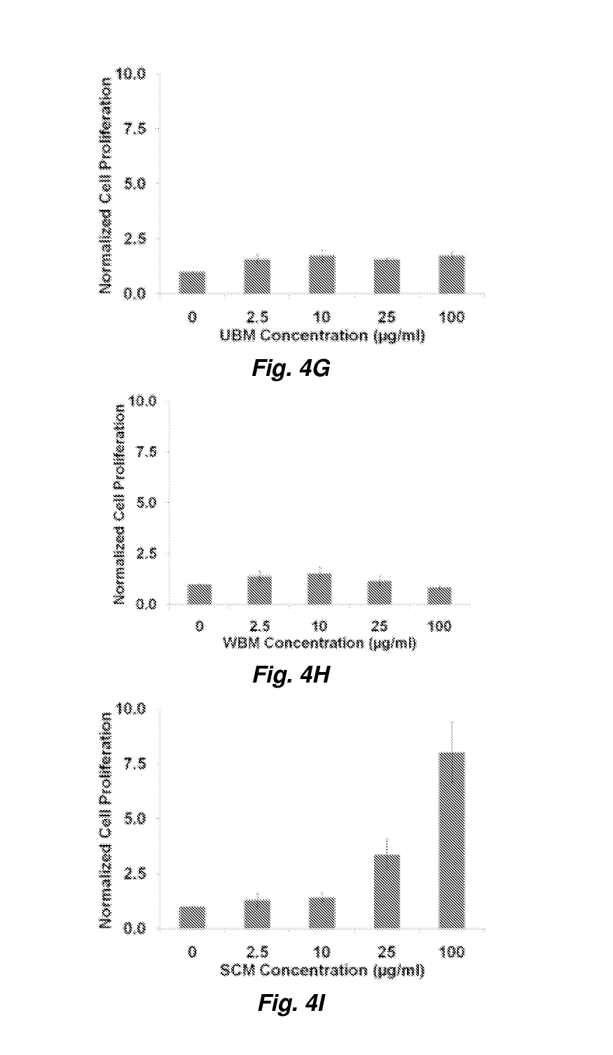

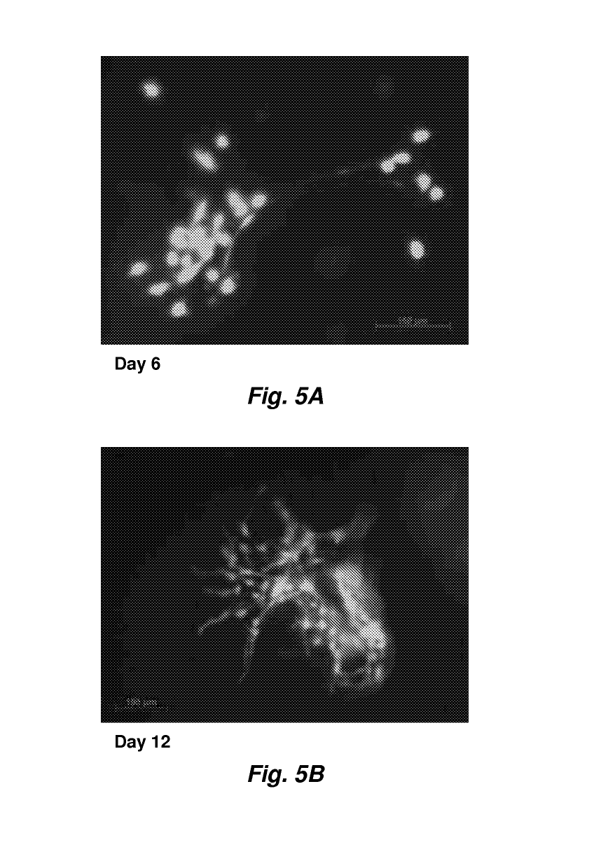

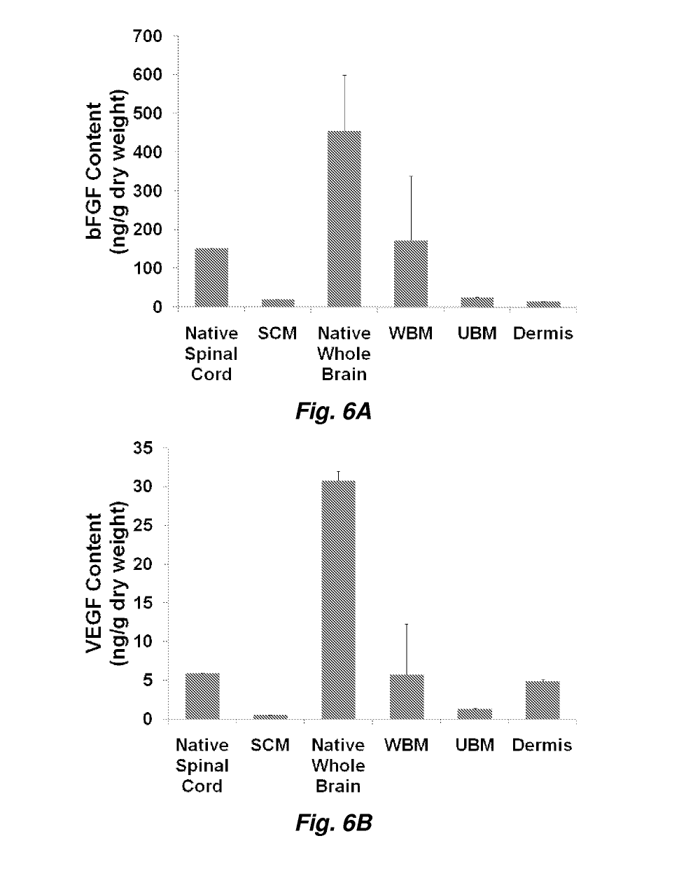

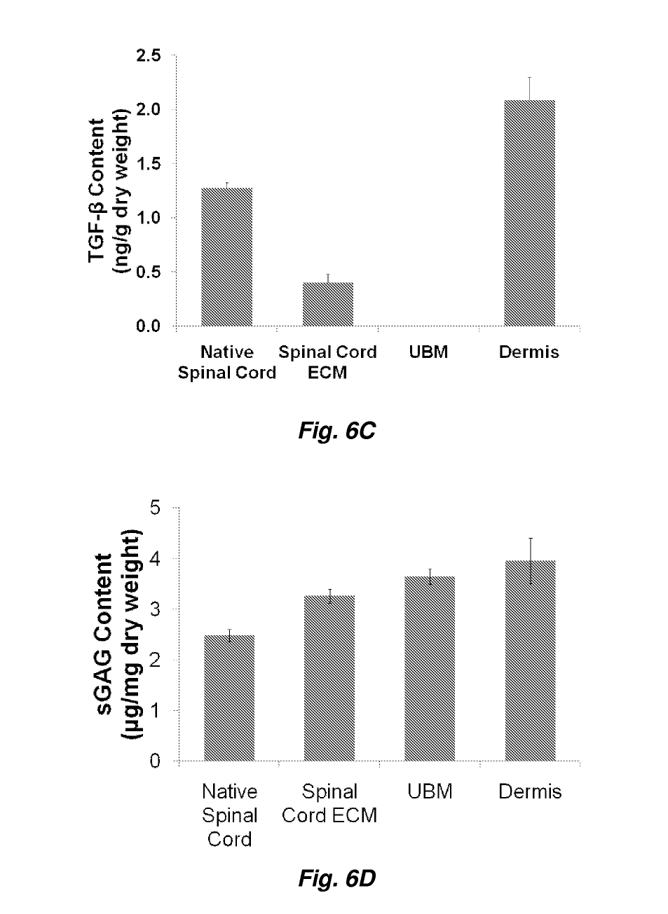

Described in this example is the characterization of the spinal cord ECM and scaffold. Prior to the step of lyophilization in Example 1, the ECM was sectioned and characterized for to cell content, or lack thereof. FIG. 1 is a digital photomicrograph showing H&E staining of CNS-derived tissues and ECM. FIG. 1C. shows native spinal cord. The nuclei are stained of cells (and a few other objects, such as granules) are stained blue, while other materials or structures are stained various shades of red, pink and orange. FIG. 1D is a digital photomicrograph showing H&E stained spinal cord ECM. The absence of punctate blue nuclei demonstrates that intact cells are absent from the section. This observation is further supported by FIG. 2B, which demonstrates the lack of DNA in spinal cord ECM as compared to native spinal cord tissue.

The growth factor content, ability to promote chemotaxis and mitogenesis of neural contributing stem cells, ability to form a gel, as well as the ability to promote neurite formation, were all measured.

Growth factor content of SCM:

bFGF: 44% of content found in native spinal cord
VEGF: 84% of content found in native spinal cord
TGF-β: 32% of content found in native spinal cord

Growth factor content of WBM:
bFGF: 17% of content found in native brain
VEGF: 0.3% of content found in native brain.

CNS stem cells have been cultured with SCM and WBM to assess potential cytotoxic, mitogenic, and chemotactic effects. The CNS stem cells investigated to date include fetal human cortical neuroepithelial progenitor (CTX) cells (commercially available from ReNeuron, Ltd., Guildford, Surrey, UK).

In brief, 2.13×10⁶ CTX cells were deposited in either UBM or SCM gel at 2.75 mg/mil fully submerged in reduced modified medium with bFGF, EGF, and 4-OHT (hydroxytamoxifen) for 6, 12, or 24 days. At each time point the medium was removed and the gels were immersed in 10 μM calcein AM which fluoresced upon intracellular cleavage to calcein, resulting in fluorescence of cytosol in live cells only which allowed neurite imaging. We have observed neurite sprouting by human neural stem cells (indicative of neural stem cell differentiation) in spinal cord ECM but not in urinary bladder ECM. Table 1 summarizes these results. Culture of human brain stem cells in spinal cord ECM gel produced cell processes sufficiently long to be called neurites, but longer processes likely to be neurites were not observed in urinary bladder ECM gel of comparable stiffness. The tabulated results indicate that there are tissue-specific effects of CNS ECM on CNS cells.

TABLE 1

| | | Most Common | Maximum Observed |
|---|---|---|---|
| Time Point | ECM Type | Observed Length | Length |
| Day 6 | Spinal Cord ECM | ~50 micron | ~200 micron |
| | Urinary Bladder ECM | ~20 micron | ~30 micron |
| Day 12 | Spinal Cord ECM | ~100 micron | ~200 micron |
| | Urinary Bladder ECM | ~20 micron | ~30 micron |
| Day 24 | Spinal Cord ECM | ~200 micron | ~400 micron |
| | Urinary Bladder ECM | ~20 micron | ~30 micron |

Additionally, as shown herein, we see that in some cases spinal cord or brain ECM modulate proliferation (mitogenesis) and migration (chemotaxis) of neural stem cells more than urinary bladder ECM, though in other cases urinary bladder ECM induces a greater response. Overall, there appear to be tissue-specific advantages for using CNS ECM for CNS applications compared to non-CNS ECMs.

### Example 3

### Characterization of ECM Gels

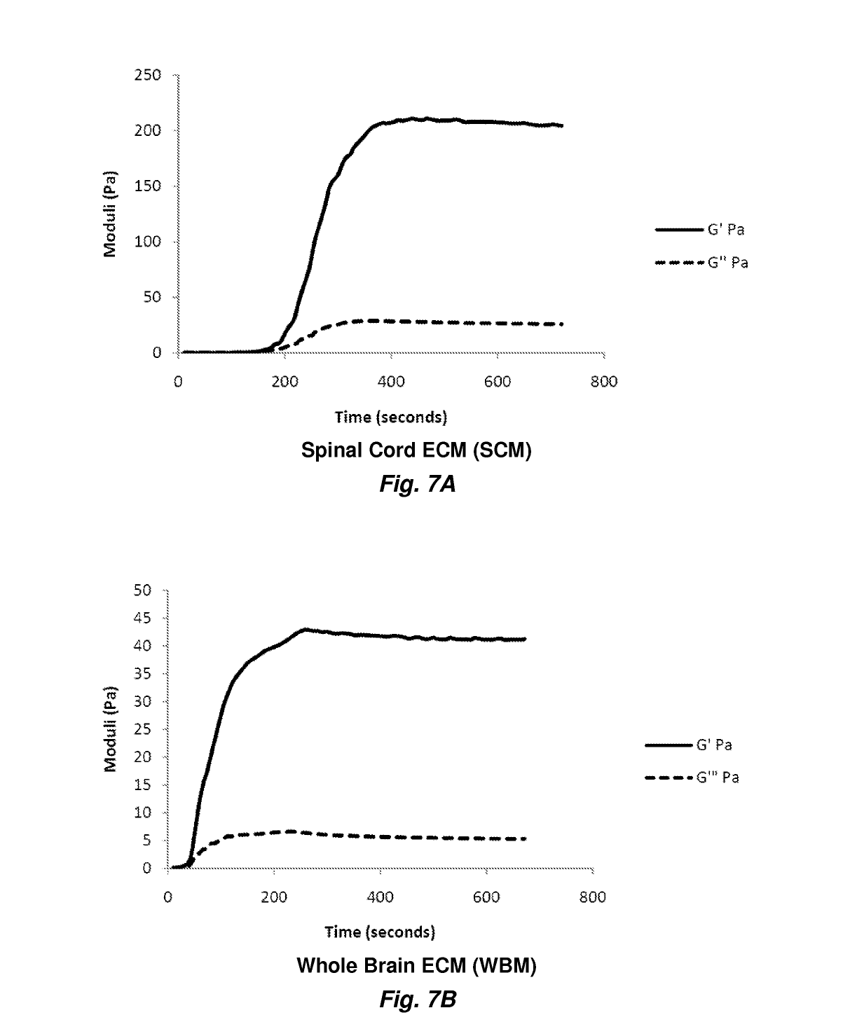

Brain ECM and Spinal Cord ECM were enzymatically digested in pepsin. In brief, Brain ECM and Spinal Cord ECM were placed into 0.5 mg pepsin/0.01N HCL solution at a concentration of 10 mg ECM powder/mL for 48-72 hours. The pH was then neutralized and the stock digest diluted to 6 mg/mL and placed on a parallel plate rheometer at 1 Pa creep and 10° C. to ensure the sample was evenly distributed between the plates. A dynamic time sweep with the parameters of 5% strain, 1 angular frequency, and 37° C. determined the time to complete gelation. Complete gelation was defined by the plateau of the G′ (elastic modulus) measurement. As shown in FIG. 7, spinal cord ECM formed a gel with a storage modulus (G′) of 190 Pa and a loss modulus (G″) of 33 Pa. FIG. 7 also shows a graph of the storage modulus (G′) and loss modulus (G″) for whole brain ECM.

### Example 4

### Treatment Using ECM Gels in an Animal Model

UBM and a combination of UBM and SCM or UBM and WBM have been injected into the cerebral and cerebellar regions of rat brain through a surgically-created cranial port. In brief, a rat was placed under deep anesthesia, at which point a small burr hole was created through the animal's skull and dura mater. At this point, the UBM was injected into the healthy cerebellum. The concentration of UBM injected into the healthy cerebellum was 8 mg/mL, at a volume of 10 μL

Surprisingly, injection of this xenogeneic ECM into healthy rat cerebellum showed no adverse effects such as acute inflammatory or adverse immune responses. Injection of UBM and WBM gel combined with neural stem cells into the lesion of a post-stroke rat cerebrum recruited host cells into the lesion space.

### Example 5

### Repair of Spinal Cord after Contusion

As proof of concept as to the usefulness of ECM products in the functional recovery after spinal cord trauma, a rat is subjected to spinal cord injury.

All rats are kept in their cages for at least 3 days between arrival and surgery. Rats are anesthetized with 50 mg/kg ketamine and 1.0 mg/kg meditomidine. This dose normally provides sufficient analgesia and reduces negative risks associated with anesthesia. If necessary, the dose is increased to 75 mg/kg ketamine with 1.0 mg/kg meditomidine to provide sufficient analgesia. The hair is shaved from the back and the area cleaned with betadine and 70% alcohol. The rat's eyes are protected from drying during the procedures using artificial tears ointment. All instruments for surgery are autoclaved for (steam) sterilization before use. Aseptic technique is used in all surgical approaches. During surgery, instruments are desinfected using hot bead sterilization (table-top). After 5 surgical sessions all instruments are steam sterilized.

Procedures During Surgery/Spinal Cord Injuries:

Rats are kept on a 37° C. heating pad. The spinal cord is exposed through laminectomy of the T7-11 vertebrae. Next, the dorsal or lateral column is transected or a 2-3 mm long segment within the dorsal/lateral column removed. See below for detail on transection injury procedures. After injury procedures, the wound/transplantation area is rinsed using sterile PBS with 0.1% gentamicin. Muscles are closed separately using 4.0 prolene sutures and the skin is closed with metal wound clips.

General Procedures Regarding Injury Surgeries:

During all surgical procedures rats are kept on a heating pad at 37+/−1° C. A laminectomy (removal of the dorsal part of the spinal vertebrae) is used to expose the spinal cord. After deep anesthesia is reached, a midline incision is made above the mid/lower thoracic spinal column. The paraspinous muscles are separated and reflected laterally to expose the T7-11 spinal vertebrae. The spinous processes and laminae are nipped away (number and level depending on the various types of injuries; specified below) with small rongeurs until the underlying spinal cord is exposed without damaging the underlying dura and spinal cord. The animal is then prepared for contusions, or injections for treatment or tracing (see below for details).

General Procedure for Contusion Injury:

The dura mater is longitudinally incised. The exposed spinal cord is contused between the T7-11 spinal vertebrae. The contusion injury is made using a computer controlled impaction device (IH impactor. The computer controls the impact speed and force of a sterilized piston to ensure reproducibility. Before placing the animal under the IH impactor and securing it for contusion injury, the forceps are carefully adjusted to level the spinal cord in the horizontal plane.

Additionally, the computer displays the impact force ensuring continued reproducibility between surgeries. Contusion force is 200 kDyne. There is <0.05% variation between these values.

ECM repair material is immediately injected in the amount of 5 uL into the center of the injury using a Hamilton syringe with an attached pulled glass needle. After injection, the needle is kept in place for an additional 3 min to prevent backflow while withdrawing. When bleeding has stopped, the wound/transplantation site is rinsed with sterile PBS with 0.1% gentamicin, the muscles is closed separately using 4.0 prolene sutures, and the skin is closed using metal wound clips.

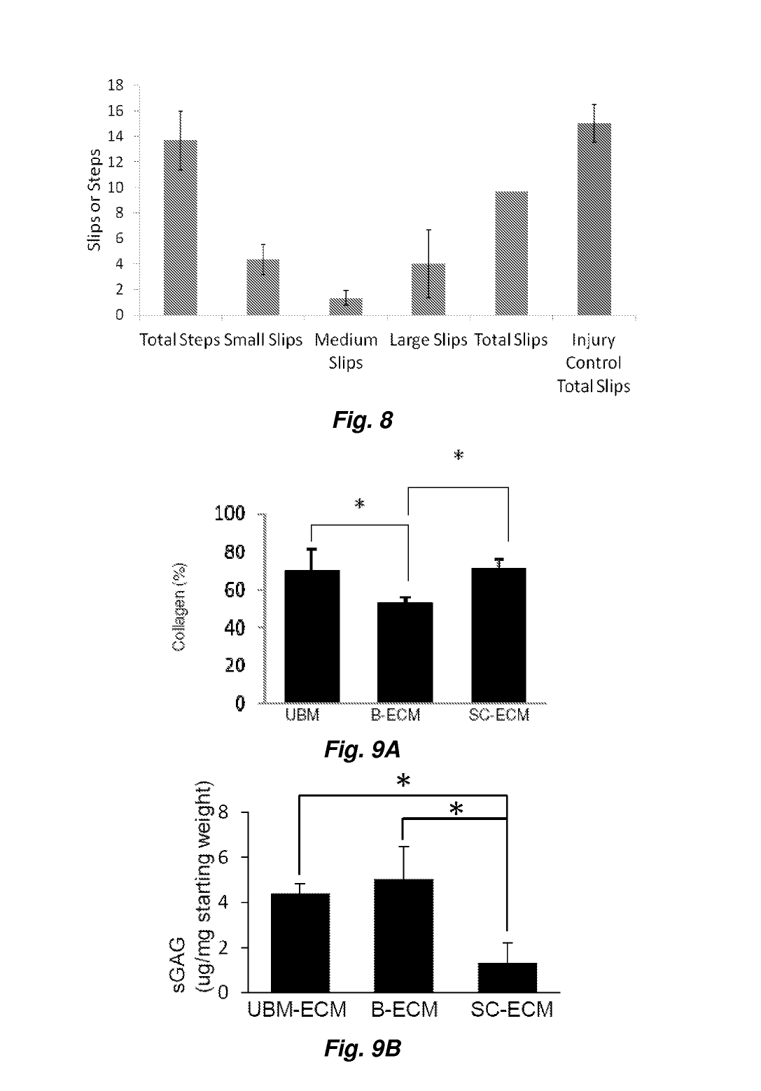

The rat was allowed to heal for four (4) weeks and was then functionally assessed. In this test, the rat walks across a 1 meter metal grid and is assessed for total number of slips (No Slip: The foot maintains attachment to the bar. Small Slip: The toes and foot slip off the bar, and dip into the space between. Medium slip: The toe and leg slips down below the bars, but so much that the fur line goes beneath the bar. Large slip: The whole leg falls between the grid, see, Pearse, D. D., et al., A. E. Marcillo, et al. (2004) “Transplantation of Schwann cells and olfactory ensheathing glia after spinal cord injury: does pretreatment with methylprednisolone and interleukin-10 enhance recovery?” J Neurotrauma 21(9): 1223-1239). As shown in FIG. 8, the total number of slips for the rat treated with UBM is statistically lower than the injury control (control data obtained from Pearse et al.). Given the superior results obtained from CNS-derived ECM material with respect to neurite growth as compared to UBM materials, as described above, equal or better results are expected from the CNS-derived ECM material.

In another example, CNS-derived ECM material is injected at a site of injury to the brain or spinal cord of a human patient in an amount effective to support repair of the patient's CNS tissue. At one or more times between one week and two years after injection, repair of the injury is monitored by imaging, and one or more tasks affected by or associated with the injury to the patient are tested.

### Example 6

### Manufacturing Decellularized Whole Brain Biological ECM Scaffolds

In one example, porcine whole brain was obtained. The whole brain tissue was transported to the laboratory on ice, wrapped in freezer paper, and stored at −80° C. until needed for ECM derivation processing. The dura mater was removed from the whole brain, and the whole brain was cut into quarters longitudinally of about 1 inch length and uniform thickness. The whole brain pieces were placed into water overnight at 4° C. and 120 rpm, to mechanically disrupt the native tissue architecture prior to decellularization. After about 18 h the whole brain pieces were removed from the water by straining onto a mesh or sieve with hole size of about 840 μm. The pieces of spinal cord were collected with forceps and placed into a flask for protease digestion with 0.02% trypsin/0.05% EDTA solution. The digestion was allowed to proceed in a water bath for 1 h at 37° C. while shaking at 120 rpm. After one hour, the solution was strained off and whole brain tissue was rinsed gently under a stream of water, detangling as required. The whole brain pieces were returned to the flask, collecting as many smaller tissue pieces as possible from the strainer using forceps. 3% Triton X-100 solution was then added to the flask to begin decellularization of the tissue, which was place upon a shaker for 1 h at 120 rpm. After one hour, the tissue was strained, rinsed, and collected. The tissue pieces were placed back into the flask and then were subjected to osmotic shock for additional decellularization. Hypertonic 1 M sucrose was added to the flask and the flask was placed on a shaker for 15 min at 200 rpm. The tissue was strained, rinsed in water, collected and combined with hypotonic solution (deionized water) and placed on shaker for 15 min at 200 rpm, to lyse any remaining cells. The decellularized tissue was again strained, rinsed in water, and reclaimed into a flask. 4% deoxycholate solution was added to the flask and placed upon a shaker for 1 h at 120 rpm. Subsequently, the tissue pieces were strained and rinsed repeatedly in type I water until all traces of surfactants (bubbles) were removed. The remaining tissue, now enriched into ECM, was collected and disinfected using a peracetic acid solution (Enviroguard MP2, 15% PAA (Source: Envirotech ETPA15MP-0045 or equivalent in a mixture of Type I water (96%) and 100% EtOH alcohol (4%) to a final PAA concentration of 0.1% wt.) at a ratio of 20:1 peracetic acid solution to weight of ECM, and shaken at 200 rpm for two hours. Following a series of rinse steps in Phosphate Buffered Saline (PBS), the ECM frozen at −20° C. and then was lyophilized until all water was removed.

Of note, routine methods of decellurization of ECM tissue, for example those methods described herein for preparing UBM or SIS material, were ineffective for the production of useful CNS tissue.

### Example 7

### Characterization of the Whole Brain ECM Scaffold

Verification of Decellularization:

Both native tissues and ECMs were sectioned, stained with H&E or DAPI, and imaged to qualitatively assess DNA content. For quantitative analysis, finely chopped ECM was digested with 0.1 mg/ml proteinase K solution for at least 24 hours, protein was removed by phenol/chloroform extraction and centrifugation, and DNA was resuspended in 3 M sodium acetate with ethanol rinses and dried. dsDNA was quantified using PicoGreen (Invitrogen) per kit instructions.

Growth Factor Content:

Finely chopped native tissues and ECMs were suspended in extraction buffer with protease inhibitors. For VEGF and bFGF, the buffer included 2 M urea and 5 mg/ml heparin. Suspensions were centrifuged at 12,000 g and the extraction process was repeated. Supernatants were dialyzed and growth factors were quantified using Quantikine immunoassay kits (R&D Systems). For NGF, the buffer included 1% NP40, 10% glycerol, and 0.5 mM sodium orthovanadate with centrifugation at 2,000 g. NGF was quantified using an Emax immunoassay kit (ProMega).

### Results: Characterization of Residual DNA in CNS ECMs.

After H&E staining, cell nuclei were visible in native spinal cord tissue but not in spinal cord ECM. Cell nuclei were also visible in native brain tissue but not in brain ECM. DAPI showed similar results in native spinal cord, spinal cord ECM, native brain, and brain ECM. DNA quantification via dsDNA extraction and PicoGreen assay showed lower concentrations of DNA in ECMs compared to native tissues, with <50 ng DNA per mg dry weight in ECMs. (See FIGS. 1-2.)

Collagen Quantification:

Collagen concentrations were determined in Brain-ECM and Spinal cord-ECM (n=3 each) using the Collagen Assay Kit (Biocolor Ltd., Carrickfergus, Co Antrim, United Kingdom) and following the manufacturer's recommended protocol. Samples were prepared by pepsin digestion at a concentration of 10 mg ECM/mL for 48 hours at room temperature.

Quantification of collagen (Types I-V) determined the total concentration remaining in Brain-ECM and Spinal Cord-ECM following decellularization. Spinal cord-ECM contained significantly higher concentrations, 703+/−47 μg collagen/mg starting weight compared to Brain-ECM, which contained 537.47+/−26.9 μg collagen/mg starting weight (p<0.01).

### Example 8

### Manufacture of Mixed Gels

A CNS ECM preparation comprising CNS-derived ECM material and synthetic or additional biological polymer compositions is provided. All materials are produced by reconstituting (hydrating) dry, powdered polymeric components with water or PBS. Alternately, the various components of the CNS ECM material are hydrated and mixed prior to gelation. Dry components are milled to a powder (approximately <1 mm average diameter and <840 μm (micron) in diameter) in a classification mill, and components are dry-mixed prior to hydration. For the CNS-derived ECM material, WBM and/or SCM is used. For the synthetic polymer component, either PEUU, PEEUU, PECUU or PCUU are used. For the additional ECM component, either UBM or SIS is used. The following mixtures are prepared with total polymeric components (mg/mL of CNS-derived ECM material+mg/mL of synthetic polymer component+mg/mL of additional ECM component) totaling 1, 10, 25, 50, 100, 150 and 200 mg/mL:
WBM (100% of total polymeric components).
SCM (100% of total polymeric components).
CNS-derived ECM material (1-99% of total polymeric components)+synthetic polymer component (1-99% of total polymeric components).
CNS-derived ECM material (1-99% of total polymeric components)+additional ECM component (1-99% of total polymeric components).
CNS-derived ECM material (1-99% of total polymeric components)+synthetic polymer component (1-99% of total polymeric components)+additional ECM component (1-99% of total polymeric components).

In an extension of this, an effective amount water-soluble active agent is either mixed into the dry mixture prior to hydration or is added to the hydrated mixture of materials.

The hydrated materials are injected or otherwise placed at a site of a wound or defect in a patient.

### Example 9

Overview of Experimental Design:

Following decellularization of porcine brain and spinal cord, brain ECM and spinal cord ECM were solubilized via pepsin digestion and methods identified to facilitate gelation. The ECM materials were analyzed for collagen and sulfated glycosaminoglycans, microstructure, and gel mechanical properties characterized by rheology and turbidimetric assays. A model cell line, N1E-115 (Bordt, S. L., et al., N1E-115 mouse neuroblastoma cells express MT1 melatonin receptors and produce neurites in response to melatonin. Biochim Biophys Acta, 2001. 1499(3): p. 257-64; Dan, C., et al., PAK5, a new brain-specific kinase, promotes neurite outgrowth in N1E-115 cells. Mol Cell Biol, 2002. 22(2): p. 567-77; and Sarner, S., et al., Phosphatidylinositol 3-kinase, Cdc42, and Rac1 act downstream of Ras in integrin-dependent neurite outgrowth in N1E-115 neuroblastoma cells. Mol Cell Biol, 2000. 20(1): p. 158-72), was used to identify the neurotrophic potential of ECMs gels in two and three dimensional culture. The assays and results were compared to previously described ECM gel manufactured from urinary bladder matrix (UBM).

ECM Biologic Scaffold Production:

Porcine brain, spinal cord, and urinary bladder were obtained from market weight animals at a local abattoir. Tissues were frozen immediately after harvesting at −80° C.), thawed completely before use, and processed independently by tissue specific methods as described above. In brief, CNS tissue was agitated (spinal cord tissue −200 RPM; brain tissue 120 RPM) in the following decellularization baths: deionized water (16 h at 4° C.; 60 rpm); 0.02% trypsin/0.05% EDTA (60 min at 37° C.; 60 RPM; Invitrogen Corp., Carlsbad, Calif., USA), 3.0% Triton X-100 (60 min) (Sigma-Aldrich Corp., St. Louis, Mo., USA); 1.0 M sucrose (15 min) (Fisher Scientific, Pittsburgh, Pa., USA); deionized water (15 min); 4.0% deoxycholate (60 min) (Sigma); 0.1% peracetic acid (Rochester Midland Corp., Rochester, N.Y., USA) in 4.0% ethanol (120 min); PBS (15 min) (Fisher); deionized water (15 min); deionized water (15 min), and PBS (15 min). Each bath was followed by rinsing of remaining tissue through a strainer with deionized water. Decellularized brain and spinal cord were lyophilized and stored dry until use.

Urinary bladder matrix (UBM) was prepared as follows. Connective tissue was removed from the serosal surface of the bladder. The tunica serosa, tunica submucosa, and majority of the tunica muscularis mucosa were mechanically delaminated, which left the basement membrane and tunica propria intact. Luminal urothelial cells were dissociated from the basement membrane by soaking the UBM in deionized water. The UBM was then agitated in 0.1% peracetic acid (Rochester Midland Corp., Rochester, N.Y., USA) in 4.0% ethanol (120 min; 300 RPM). Then the UBM was rinsed in a series of phosphate buffered saline and deionized water to ensure thorough peracetic acid removal.

ECM Digestion and Solubilization:

Lyophilized and comminuted B-ECM (20 mesh), SC-ECM (20 mesh or hand cut), or UBM (20 mesh or hand cut; 400 μm-1000 μm largest particle dimension as measured by mesh diameter or ruler) were placed into a 0.01 N HCl solution containing 0.1 mg/mL pepsin at a concentration of 10 mg ECM/mL and stirred at room temperature for 48 h (See, e.g., US 20080260831). After 48 h, brain ECM, spinal cord ECM, and UBM were completely digested to form a pre-gel solution (pH-2). The pre-gel ECM solution was brought to pH 7.4 using 0.01N NaOH and diluted to the desired volume/concentration using 10× and 1×PBS. Pepsin is irreversibly inactivated at pH above 7.5.

Collagen and sGAG Quantification:

Collagen concentration of the pre-gel ECM solution was determined with the Sircol Assay Kit (Biocolor Ltd., UK) following the manufacturer's recommended protocol. The assay was repeated four times with independent samples in triplicate.

sGAG Quantification:

Collagen concentration of the pre-gel ECM solution was determined for samples from independent production batches with the Sircol Assay Kit (Biocolor Ltd., UK) following the manufacturer's recommended protocol (n=4 in duplicate or triplicate). Sulfated glycosaminoglycan (sGAG) concentrations were determined using digested ECM at a concentration of 50 mg ECM/ml with 0.1 mg/ml proteinase K (Sigma) in buffer (10 mM Tris-HCl, pH 8.0, 100 mM NaCl, 25 mM EDTA for 48-72 hours at 50° C.) using the Blyscan Sulfated Glycosaminoglycan Assay Kit (Biocolor Ltd., UK) and following the manufacturer's recommended protocol (n=3 in duplicate or triplicate).

Scanning Electron Microscopy:

Scanning electron microscopy was used to examine the surface topography of brain, spinal cord, and UBM ECM gels. Five hundred micron thick gels were prepared and then fixed in cold 2.5% glutaraldehyde for 24 hours followed by three 30 minute washes in 1×PBS. Gels were dehydrated in a graded series of alcohol (30, 50, 70, 90, 100% ethanol) for 30 minutes per wash, and then placed in 100% ethanol overnight at 4° C. Gels were washed 3 additional times in 100% ethanol for 30 minutes each and critical point dried using a Leica EM CPD030 Critical Point Dryer (Leica Microsystems, Buffalo Grove, Ill., USA) with carbon dioxide as the transitional medium. Gels were then sputter-coated with a 4.5 nm thick gold/palladium alloy coating using a Sputter Coater 108 Auto (Cressington Scientific Instruments, UK) and imaged with a JEOL JSM6330f scanning electron microscope (JEOL, Peabody, Mass., USA)

Turbidity Gelation Kinetics:

The pre-gel solution was diluted to 6 mg/mL and maintained on ice at 4° C. until placed into a 96 well plate (100 uL/well). The plate was immediately transferred to a spectrophotometer (Molecular Devices) preheated to 37° C., and absorbance was measured at 405 nm every 2 minutes for 50 minutes. The time required to reach 50% and 95% maximum absorbance was denoted as t_{1/2} and t₉₅, respectively, the lag phase, t_{tag}, calculated by extrapolating the linear portion of the curve, and the turbidimetric speed, S, of gelation was determined by calculating the growth portion slope of the curve normalized to absorbance as seen in equation 1. The assay was repeated three times with independent samples in triplicate.

$$Normalized Absorbance = \frac{A - {A}_{o}}{{A}_{ma x} - {A}_{o}} \qquad Equation 1$$

Rheological Measurements:

The pH of the pre-gel solution was neutralized to 7.4 and the solution diluted to 4, 6, or 8 mg/mL. The diluted pre-gel solution was then placed on a 40 mm parallel plate rheometer (AR 2000, TA Instruments) at 1 Pa creep and 10° C. to ensure even distribution of pre-gel solutions between the plates. A dynamic time sweep was run with the parameters of 5% strain, 1 rad/s (0.159 Hz), and increasing temperature from 10° C. to 37° C., unless otherwise noted. The storage modulus (G′), and the loss modulus (G″) were determined by the steady state plateau of the curve. The assay was repeated three times with independent samples in triplicate.

N1E-115 ECM Cytocompatibility and Two Dimensional Neurite Extension:

N1E-115 mouse neuroblastoma cells (ATCC No. CRL 2263), a commonly used experimental cell line to examine neurotropic potential and differentiation, were cultured in DMEM (Sigma) with 10% fetal bovine serum (FBS; Thermo Fisher Scientific, Waltham Mass., USA)/1% pen/strep (Sigma) at a concentration of 100,000 cells per well in 12 well plate prior to the addition of ECM. B-ECM, SC-ECM, or UBM-ECM digest was added after cell attachment at a concentration of 100 ug ECM/mL. Following 18-24 hrs in culture with ECM, the medium was removed and 4 uM calcein-AM and 4 uM ethidium homodimer-1 was added to each well to evaluate cytotoxicity. Live cells that hydrolyze membrane-permeable calcein-AM, but not ethidium homodimer-1, fluoresce in green and dead cells that bind and fluorescence activate ethidium homodimer-1, but not calcein-AM, fluoresce in red.

The effects of the B-ECM, SC-ECM, and UBM-ECM pre-gel digest effect upon N1E-115 neurite outgrowth were independently evaluated and used as an indicator of neurotrophic potential of the remaining bioactive molecules in ECM materials after enzymatic degradation. N1E-115 cells in DMEM with 10% FBS/1% pen strep were seeded at a density of 5,000 cells/well in a 24 well plate. After incubation for 24 h, the media was removed and replaced with DMEM with 2.5% FBS, which promoted low levels of neurite extension and allowed changes in response to ECM to be quantified. Neutralized ECM pre-gel solutions at concentrations of 10 μg/mL and 100 μg/mL were added after cell attachment and cells were incubated for 48 h. N1E-115 cells were fixed with 2% paraformaldehyde for 20 minutes at room temperature. Attached cells were stained with DAPI for nuclei and Alexa Fluor phalloidin 488 (Invitrogen) for F-actin filaments. Three images at 200× magnification were taken per well. The number of cells with neurite extensions were manually counted. The longest neurite of each cell was measured in pixels using ImageJ (NIH). The outgrowth assay was repeated six times per condition. Neurites were denoted as cell processes that extended a minimum length of twice the diameter of the cell body. Image stacks were imported into Imaris software (Bitplane, South Windsor Conn., USA) for 3-D visualization.

Neurite Extension in Three Dimensional Culture:

N1E-115 cells were maintained in 10% FBS DMEM media at 37° C. B-ECM, SC-ECM, or UBM-ECM hydrogels (1 ml) were cast with a cell density of 500,000 cells/hydrogel and a concentration of 6 mg ECM/ml and after gelation for 1 hr at 37° C. in a non-humidified incubator. All hydrogels were then cultured for 24 hours in DMEM supplemented with 10% FBS/1% pen/strep. Serum concentration was reduced to 0% FBS and cells cultured for an additional 2 or 7 days. The hydrogels were then fixed with 4% paraformaldehyde (Fisher), stained for F-actin, and imaged using multiphoton confocal microscopy to visualize three-dimensional cell morphology inside the hydrogel scaffolds.

Hydrogels cultured with N1E-115 cells were stained with 0.1% Alexafluor 488 Phalloidin (Invitrogen) for 2 hours and submerged in PBS solution in a hanging drop slide and coverslipped. To visualize the axonal trajectories in three dimensions, the slide was mounted beneath an Olympus FV1000 multiphoton system. The system was equipped with a Chameleon ultra-diode-pumped laser, and a 25×XL Plan N objective with a N.A. of 1.05 and a field of view of 500 um. The excitation wavelength was chosen at 830 nm at a 6% laser transmissivity. The sampling speed was set to 2 μs/pixel with a 2 line kalman filter, and the scanning had an incremental z-step of 1 μm. Image stacks were then compiled into a maximum intensity z-projection in ImageJ.

Statistical Analysis:

An independent Student's t-test was used to detect the effect of ECM pre-gel digests on N1E-115 differentiation compared to the no ECM control (p<0.05). A one-way ANOVA was used for all other statistical analysis (p<0.05). All statistical analysis used SPSS Statistical Analysis Software (SPSS, IBM, Chicago, Ill., USA).

### Results

Collagen and sGAG Quantification:

Collagen concentration of B-ECM was 537.47±26.9 μg collagen/mg dry weight, which was less than SC-ECM and UBM-ECM, which were of 703.24±47.3 and 702.54±113.46 μg collagen/mg dry weight, respectively (p<0.01) (FIG. 9A). B-ECM and UBM-ECM had higher sGAG concentrations, 5.05±1.41 (p<0.009) and 4.41±0.39 (p<0.02) μg sGAG/mg dry weight, respectively, compared to SC-ECM, which was 1.32±0.87 μg sGAG/mg dry weight (FIG. 9B).

Qualitative Assessment:

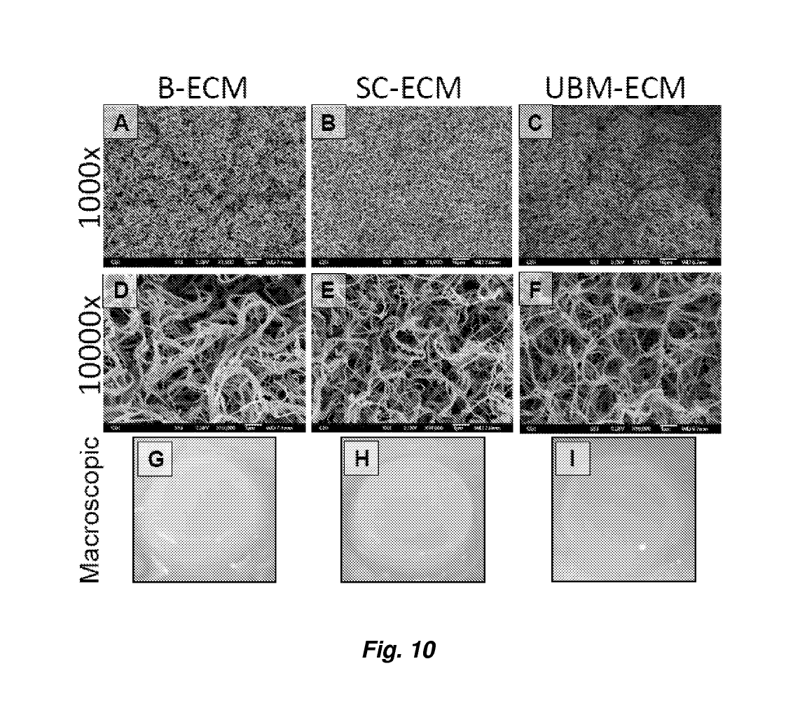

B-ECM, SC-ECM, and UBM-ECM pre-gel solutions polymerized to form a hydrogel at physiologic pH (7.4) and temperature (37° C.). Qualitatively, SC-ECM hydrogels were more rigid than B-ECM and UBM-ECM hydrogels (FIG. 10G-I).

Scanning Electron and Multiphoton Microscopy:

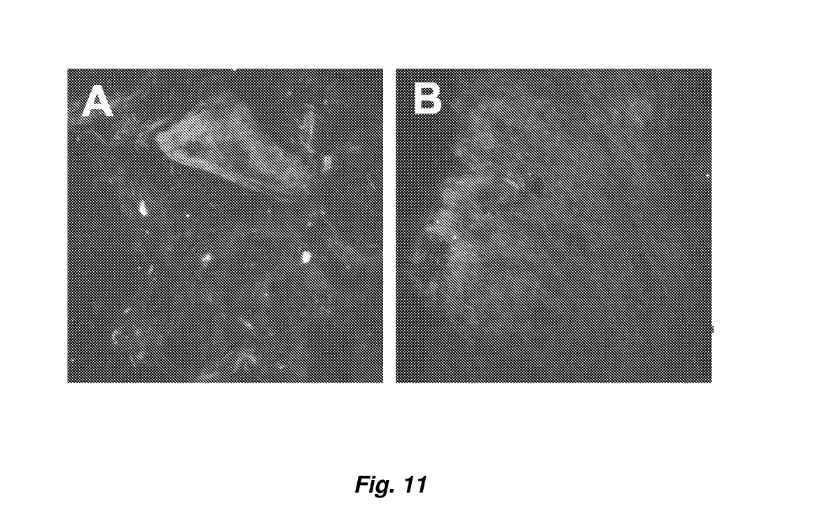

SEM micrographs showed dense, moderately organized collagen fibrils in B-ECM, SC-ECM, and UBM-ECM hydrogels (FIG. 10A-F). B-ECM contained the thickest fibrils (FIG. 10D), while SC-ECM hydrogels contains the densest (FIG. 10E). Multiphoton imaging showed the collagen structure within the B-ECM and SC-ECM hydrogels (FIG. 11). SC-ECM and UBM-ECM hydrogels contained moderately organized collagen fibers, while B-ECM contained dense clusters of randomly distributed collagen fibers.

Turbidimetric Gelation Kinetics:

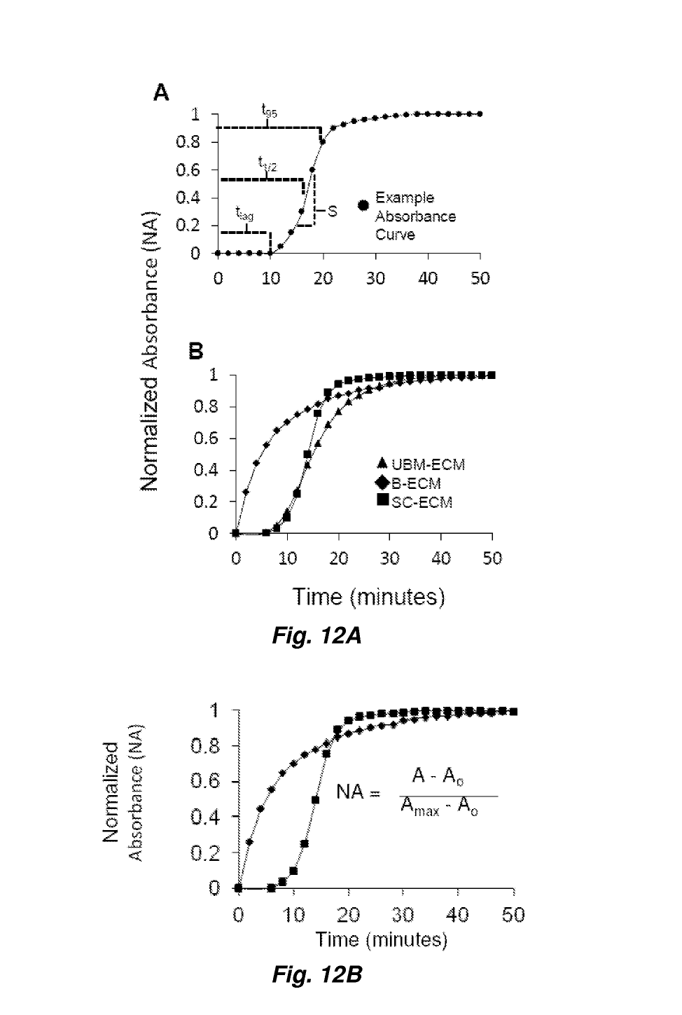

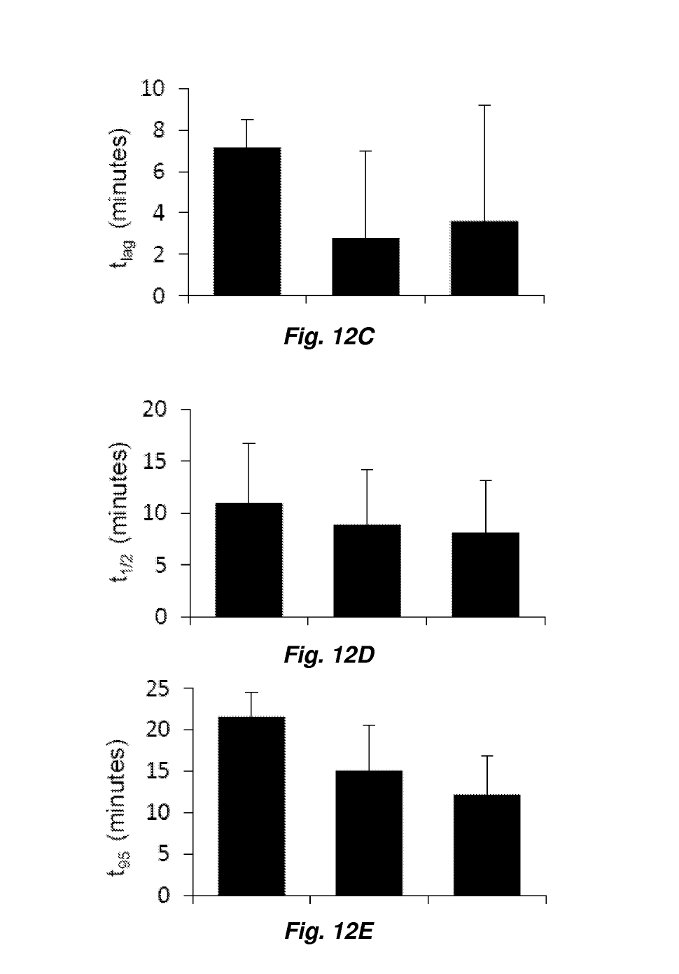

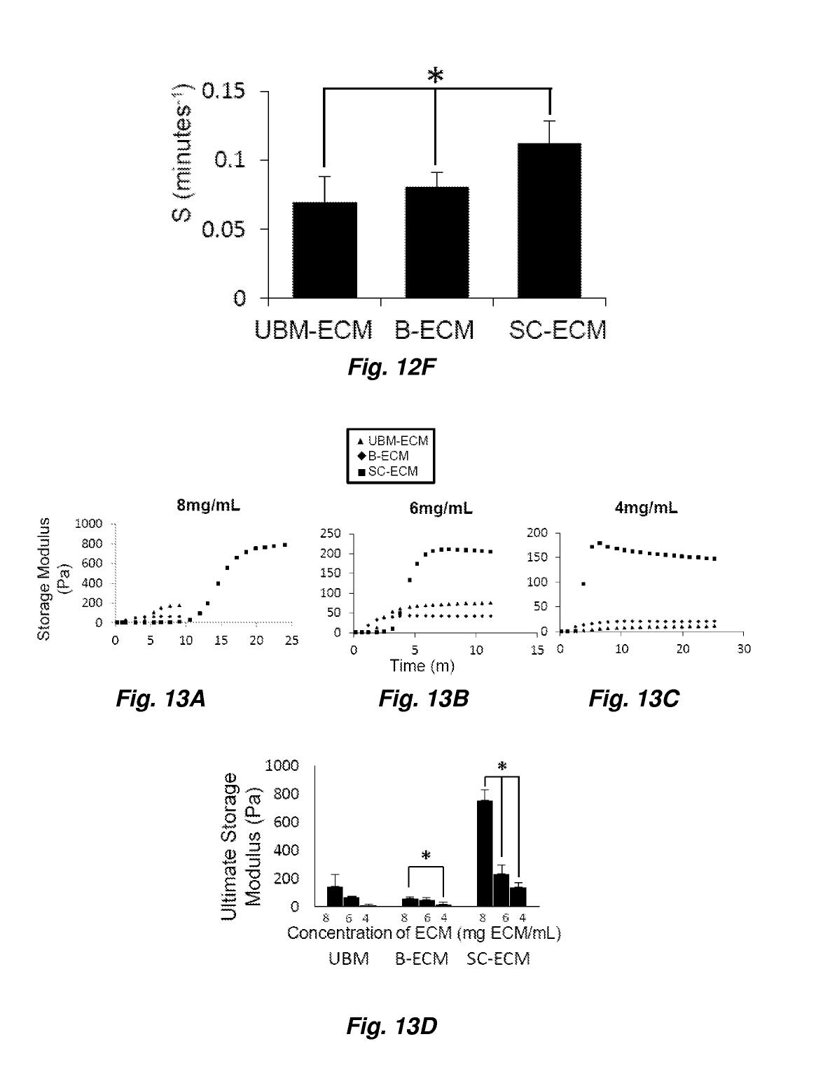

Differences in gelation kinetics between hydrogel forms of B-ECM, SC-ECM, and UBM-ECM hydrogels were evaluated using a normalized absorbance (FIG. 12A) to define the lag phase, times to reach half and 95% of the final turbidity, and speed to reach complete gelation using turbidimetric gelation kinetics as previously described (Table 2) (Freytes, D. O., et al., Preparation and rheological characterization of a gel form of the porcine urinary bladder matrix. Biomaterials, 2008. 29(11): p. 1630-7 and Gelman, R. A., et al., Collagen fibril formation. Evidence for a multistep process. J Biol Chem, 1979. 254(1): p. 180-6). For SC-ECM and UBM-ECM hydrogels, turbidimetric gelation kinetics showed a sigmoidal shape, whereas B-ECM showed a hyperbolic shape (FIG. 12B). Differences observed in kinetic curve shapes translated to a longer lag phase (t_{tag}; FIG. 12C) for UBM-ECM hydrogels (7.16±1.35 min) than B-ECM (2.79±4.22 min) or SC-ECM hydrogels (3.62±5.57 min). The time required to reach the half of the final turbidity (t_{1/2}; FIG. 12D) was also higher for UBM-ECM (11.00±5.75 min.) than B-ECM (8.87±5.40 min) or SC-ECM hydrogels (8.15±5.07 min.). The time required to reach 95% of the final turbidity (t₉₅; FIG. 12E) was also highest for UBM-ECM hydrogels (21.57±2.93 min) as compared to B-ECM (15.08±5.48 min) and SC-ECM hydrogels (12.24±4.63 min). The velocity to complete gelation (S; FIG. 12F) was faster in SC-ECM (0.11±0 0.02 min⁻¹) hydrogel than for B-ECM (0.08±0.01 min⁻¹) and UBM-ECM (0.07±0.02 min⁻¹) (p<0.05) hydrogels, suggesting that once the hydrogel assembly begins SC-ECM hydrogels reach the steady state plateau faster than B-ECM or UBM-ECM hydrogels.

Rheological Measurements:

The storage modulus (G′) and the loss modulus (G″) for both B-ECM, SC-ECM, and UBM-ECM hydrogels changed over time as the sample temperature raised from 10° C. to 37° C. Sigmoidal storage and loss moduli curves showed increasing maximum storage modulus, maximum loss modulus, and time to complete gelation with concentration (FIG. 13A-C). SC-ECM hydrogels had the largest storage modulus at all hydrogel concentrations (FIG. 13D). At 8 mg/mL, the storage modulus for SC-ECM, 757±74.87 Pa, was higher than both UBM-ECM and B-ECM hydrogels (p<0.05), which showed storage moduli of 143.8±84.1 Pa and 61.75±10.97 Pa, respectively. While B-ECM hydrogels have lower storage moduli than SC-ECM and UBM-ECM hydrogels, B-ECM hydrogels at a concentration of 6 mg/mL reached the steady state plateau in 2.4±1.25 min., which was faster than UBM-ECM and SC-ECM, which had times of 8.47±1.71 min. and 7.0±3.56 min., respectively. Table 2 summarizes the storage modulus, loss modulus, and time to complete gelation for each ECM hydrogel at concentrations of 4, 6, and 8 mg/mL.

TABLE 2

| | B-ECM | | | SC-ECM | | | UBM-ECM | | |
|---|---|---|---|---|---|---|---|---|---|
| | (Average (STDEV)) | | | (Average (STDEV)) | | | (Average (STDEV)) | | |
| Rheology | 4 mg | 6 mg | 8 mg | 4 mg | 6 mg | 8 mg | 4 mg | 6 mg | 8 mg |
| Storage | 20.31 | 49.9 | 61.75 | 138.5 | 235.5 | 757 | 11.43 | 72.78 | 143.8 |
| Modulus | (15.96) | (16.81) | (10.97) | (33.81) | (63.11) | (74.87) | (4.9) | (2.17) | (84.1) |
| (G′; Pa) | | | | | | | | | |
| Loss Modulus | 2.6 | 9.44 | 10.15 | 16.31 | 37.51 | 93.61 | 1.4 | 10.14 | 19.31 |
| (G″; Pa) | (1.94) | (4.64) | (1.8) | (4.93) | (11.39) | (10.9) | (0.59) | (0.48) | (12.27) |
| Complete | 34.8 | 2.4 | 8.33 | 11.7 | 7.0 | 28.97 | 52.53 | 8.47 | 19.8 |
| gelation | (28.87) | (1.25) | (2.76) | (5.63) | (3.56) | (4.68) | (2.15) | (1.71) | (19.1) |
| (Time; min) | | | | | | | | | |

| | | | UBM-ECM |
|---|---|---|---|
| | B-ECM (Average (STDEV)) | SC-ECM (Average (STDEV)) | (Average (STDEV)) |
| Turbidity | 6 mg | 6 mg | 6 mg |
| Speed | 0.8 (0.1) | 0.11 (0.02) | 0.07 (0.02) |
| (S; min−1) | | | |
| 50% gelation | 8.87 (5.40) | 8.15 (5.06) | 11.00 (5.75) |
| (t_{1/2}; min) | | | |
| 95% gelation | 15.08 (5.48) | 12.23 (4.63) | 21.57 (2.93) |
| (t₉₅; min) | | | |
| Lag Phase | 2.79 (4.22) | 3.62 (5.57) | 7.16 (1.35) |
| (t_{lag}; min) | | | |

N1E-115 ECM Cytocompatibility and Two Dimensional Neurite Extension:

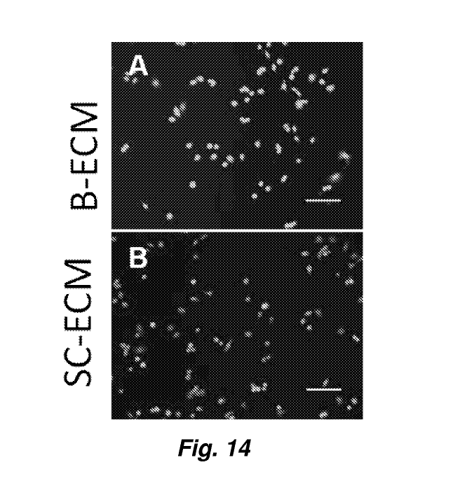

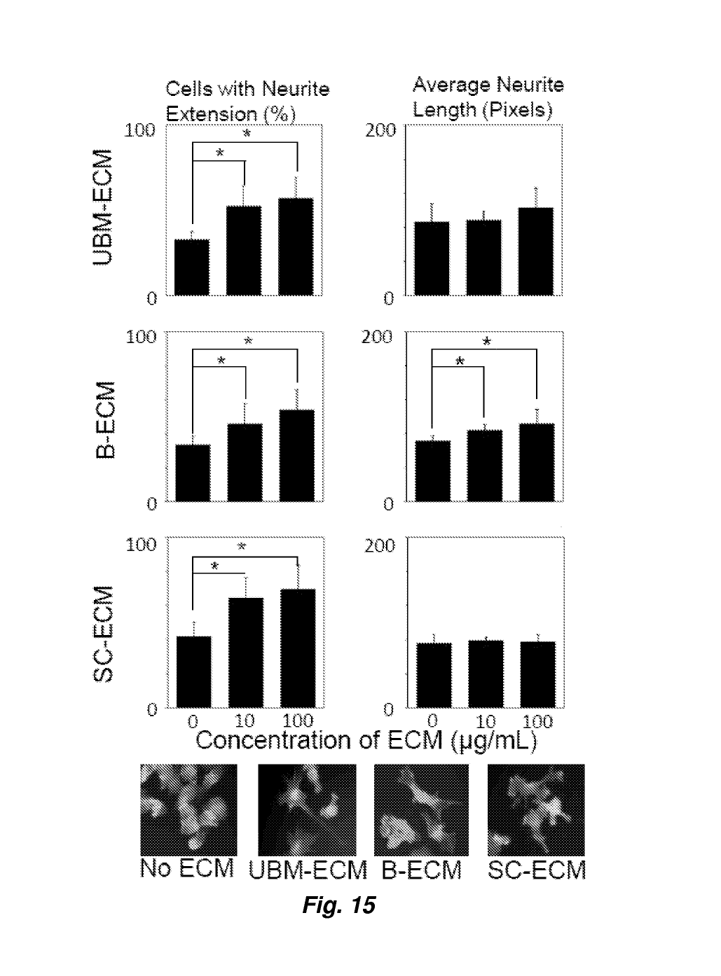

The live/dead assay showed all ECMs to be non-cytotoxic for N1E-115 cells (FIG. 14). B-ECM, SC-ECM, and UBM-ECM pre-gel solutions increased the number of cells extending neurites at concentrations of 10 and 100 ug ECM/mL compared to cells cultured without ECM digest. At 100 μg ECM/mL SC-ECM digest promoted the highest percentage of differentiation, with 69.73±13.98% of the cells extending neurites, whereas UBM-ECM and B-ECM promoted, 57.35±12.1% and 54.3±11.74% respectively. At 10 and 100 μg ECM/mL B-ECM, SC-ECM, and UBM-ECM increased the percentage of cells with neurite extensions compared to the buffered control; however B-ECM was the only scaffold that showed an increase in neurite extension length for both 10 and 100 μg ECM/ml compared to cells cultured without ECM (FIG. 15).

Neurite Extension in Three Dimensional Culture:

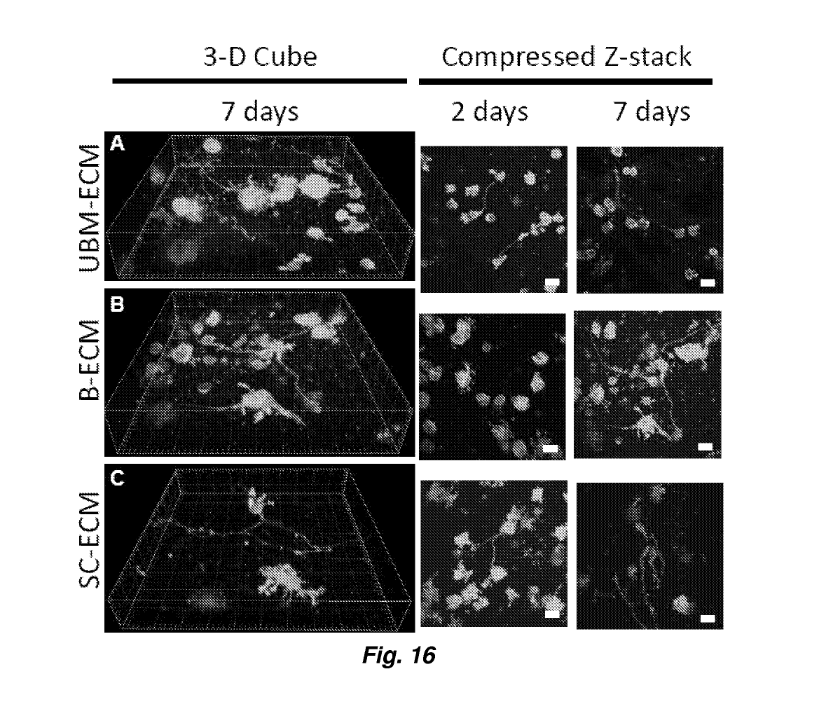

B-ECM, SC-ECM, and UBM-ECM supported three-dimensional neurite extensions at 2 and 7 days post removal of serum (FIG. 16). N1E-115 cells seeded in B-ECM hydrogels promoted a short arborizing growth pattern at two days, while SC-ECM and UBM-ECM hydrogels induce unipolar extensions. By 7-days, all ECM hydrogels promoted unipolar or bipolar extensions (FIG. 16).

### Discussion

Biologic scaffolds have been used to promote constructive tissue remodeling and functional restoration following injury (Badylak, S. F., The extracellular matrix as a biologic scaffold material. Biomaterials, 2007. 28(25): p. 3587-93). The present study shows that biologic scaffolds composed of porcine brain and spinal cord can be processed to form hydrogels that retain ECM-specific functionalities. These CNS-ECM hydrogels are cytocompatible and support differentiation of N1E-115 cells, as evidenced by neurite extension (FIGS. 16 and 17). Furthermore, the results of this study show that at comparable ECM concentrations, hydrogel forms of ECM derived from different tissues have distinctive composition and biomechanical properties, and demonstrate tissue-specific advantages for matching ECM origin with the predominant tissue type at a wound site (FIGS. 9, 13, and 15).

B-ECM and SC-ECM hydrogels, while derived by similar decellularization methods from their source tissue, have unique biochemical compositions, mechanical properties, and neurotrophic potential. The increase in neurite length for N1E-115 cells in response to B-ECM shows a tissue specific effect of B-ECM hydrogels on a brain derived cell line. CNS-ECM demonstrated neurotrophic potential in the solubilized form and supported considerable three-dimensional neurite growth and extension in the re-polymerized hydrogels, which suggests the molecular constituents of the source ECM play an important role in the bioactivity of these scaffolds. Support of three-dimensional neurite extension by CNS-ECM hydrogels also suggests the hydrogels provide the scaffolding necessary to promote in vivo axonal repair.

Although ECM biologic scaffolds from both the brain (Crapo, P. M., et al., Biologic scaffolds composed of central nervous system extracellular matrix. Biomaterials, 2012. 33(13): p. 3539-47; DeQuach, J. A., et al., Decellularized porcine brain matrix for cell culture and tissue engineering scaffolds. Tissue Eng Part A, 2011. 17(21-22): p. 2583-92; and Ribatti, D., et al., Angiogenic response induced by acellular brain scaffolds grafted onto the chick embryo chorioallantoic membrane. Brain Res, 2003. 989(1): p. 9-15) and spinal cord (Crapo, P. M., et al., Biomaterials, 2012. 33(13) and Guo, S. Z., et al., Preparation of the acellular scaffold of the spinal cord and the study of biocompatibility. Spinal Cord, 2010. 48(7): p. 576-81) have been developed for CNS tissue repair, the conformation of the harvested ECM limits the therapeutic utility of these materials. Anatomic defects, which result from common pathologic processes in the CNS such as spinal cord contusions, traumatic brain injury, and ischemic stroke, can be further complicated by secondary degeneration and liquefactive tissue necrosis resulting in irregular and unpredictable three-dimensional lesion volumes. CNS-ECM pre-gel solutions capable of conformation to existing geometric conditions and delivery by minimally invasive injection techniques are desirable materials to aid in tissue remodeling after CNS injury where opening the skull or spine is not preferred or available as a therapeutic option.

One plausible explanation for the limited investigation and use of ECM technologies for treatment of CNS injury could be the prevalence of single layer (Badylak, S. F., et al., Esophageal reconstruction with ECM and muscle tissue in a dog model. J Surg Res, 2005. 128(1): p. 87-97 and Badylak, S., et al., Resorbable bioscaffold for esophageal repair in a dog model. J Pediatr Surg, 2000. 35(7): p. 1097-103) or multilaminate sheet biologic scaffolds (Freytes, D. O., et al., Biaxial strength of multilaminated extracellular matrix scaffolds. Biomaterials, 2004. 25(12): p. 2353-61 and Aurora, A., et al., Commercially available extracellular matrix materials for rotator cuff repairs: state of the art and future trends. J Shoulder Elbow Surg, 2007. 16(5 Suppl): p. S171-8). Two-dimensional constructs have distinct advantages for numerous applications but have limited utility in the CNS due to three-dimensional tissue architecture and the required method of open-site delivery. The potential use of biologic scaffold materials in the CNS would be simplified and more clinically translatable as an injectable formulation.

Injectable formulations of various ECM scaffolds have been partially characterized in the form of hydrogels and are most commonly derived from non-neural tissues including muscle, pericardium, urinary bladder, and small intestinal submucosa (Crapo, P. M. et al., Small intestinal submucosa gel as a potential scaffolding material for cardiac tissue engineering. Acta Biomater, 2010. 6(6): p. 2091-6; Dequach, J. A., et al., Injectable skeletal muscle matrix hydrogel promotes neovascularization and muscle cell infiltration in a hindlimb ischemia model. Eur Cell Mater, 2012. 23: p. 400-12; Seif-Naraghi, S. B., et al., Design and characterization of an injectable pericardial matrix gel: a potentially autologous scaffold for cardiac tissue engineering. Tissue Eng Part A, 2010. 16(6): p. 2017-27; and Freytes, D. O., et al., Preparation and rheological characterization of a gel form of the porcine urinary bladder matrix. Biomaterials, 2008. 29(11): p. 1630-7), among others. ECM hydrogels contain properties necessary for delivery to the CNS in a minimally invasive and injectable fashion, including, but not limited to, controlled polymerization (Johnson, T. D., et al., Tailoring material properties of a nanofibrous extracellular matrix derived hydrogel. Nanotechnology, 2011. 22(49): p. 494015), ability to conform to the injection site (Seif-Naraghi, S. B., et al., Design and characterization of an injectable pericardial matrix gel: a potentially autologous scaffold for cardiac tissue engineering. Tissue Eng Part A, 2010. 16(6): p. 2017-27), and mechanical properties comparable to neural tissue (Freytes, D. O., et al., Preparation and rheological characterization of a gel form of the porcine urinary bladder matrix. Biomaterials, 2008. 29(11): p. 1630-7; Engler, A. J., et al., Matrix elasticity directs stem cell lineage specification. Cell, 2006. 126(4): p. 677-89; and Stuart, K. et al., Characterization of gels composed of blends of collagen I, collagen III, and chondroitin sulfate. Biomacromolecules, 2009. 10(1): p. 25-31). Use of CNS-ECM hydrogels for CNS repair applications is plausible, as the ECM molecules from which the hydrogels are composed were secreted by the resident cells and are likely optimized for neural tissue support. Previous studies suggest that there may be preferential effects upon cell viability and site appropriate cell differentiation for tissue specific ECM scaffolds.

As the contribution of individual biochemical components to ECM hydrogel mechanics is partially unknown, quantifying the collagen and sGAG concentrations provides insight into hydrogel rheologic properties. The shorter polymerization time and smaller storage modulus of B-ECM hydrogel compared to SC-ECM hydrogel (FIG. 13) may result from higher sGAG concentrations found in B-ECM hydrogels (FIG. 9B). Concentration of sGAGs has been shown to alter gelation kinetics and mechanical properties of hydrogels. While UBM-ECM hydrogel has similar concentrations of sGAGs compared to B-ECM hydrogel (FIG. 13), the increased storage modulus could be due to a possible increased ratio of collagen I to collagen III in UBM-ECM hydrogel, which may contribute to an increase in storage modulus. The increased storage modulus of SC-ECM hydrogel compared to UBM-ECM hydrogel (FIG. 13D), may result from relatively lower sGAG concentrations found in SC-ECM. Although collagen and sGAGs impact hydrogel mechanical properties, further studies are needed to determine which molecules present in the ECM hydrogels contribute to polymerization.

The mechanical properties of SC-ECM hydrogels were similar to compositions previously shown to support neuronal differentiation of stem cells (FIG. 13) (Engler, A. J., et al., Matrix elasticity directs stem cell lineage specification. Cell, 2006. 126(4): p. 677-89), and it is therefore plausible that the rheologic and turbidimetric properties of CNS-ECM hydrogels, which can be manipulated through ECM concentration, can influence the differentiation of endogenous or therapeutically administered stem cells following CNS injury. The range of gelation kinetics and storage moduli of CNS-ECM hydrogels allows for hydrogels to be created with tailored in vivo pre-polymerization lag time, final storage modulus, and rate of polymerization. Altering these parameters may enhance not only the ability of the gels to modulate stem cell behaviors but also the cell and drug delivery properties of CNS-ECM hydrogels.

Bioactive factors retained in the ECM hydrogels following digestion were neurotrophic as evident by the formation of N1E-115 cell neurite extensions when cultured in the presence of ECM digests. In addition to the percentage of cells with neurite extension, B-ECM digest increased the length of N1E-115 neurite extensions in two-dimensional culture. This effect was not seen in UBM-ECM or SC-ECM digest, a possible indication of a tissue-specific effect of B-ECM upon these brain derived cells (Price, A. P., et al., Development of a decellularized lung bioreactor system for bioengineering the lung: the matrix reloaded. Tissue Eng Part A, 2010. 16(8): p. 2581-91 and Amano, T., E. et al., Neurotransmitter synthesis by neuroblastoma clones (neuroblast differentiation-cell culture-choline acetyltransferase-acetylcholinesterase-tyrosine hydroxylase-axons-dendrites). Proc Natl Acad Sci USA, 1972. 69(1): p. 258-63). As organs have a unique ECM composition and structure based upon physiologic needs, ECM scaffolds homologous to the injury site may contain bioactive components unique to the organ from which the ECM was harvested. As the ECM is solubilized and digested, an array of proteins and peptides will be generated based on the molecules representative of that organ's ECM. Thus, an ECM hydrogel derived from the solubilized and digested components of the CNS will contain a unique composition of molecular constituents specific to CNS-ECM.

Although the present method, apparatus, and system have generally been described in terms of specific embodiments and implementations, it is not limited thereto. The examples provided herein are illustrative, and other variations and modifications are possible and contemplated. The foregoing specification is intended to cover all such modifications and variations.

1. A method of manufacturing a decellularized Central Nervous System (CNS) Extracellular Matrix (ECM) material, comprising:
freezing CNS tissue;
thawing the tissue;
digesting the tissue with a protease;
incubating the tissue in a solution comprising a detergent;
osmotically shocking the tissue by incubating the tissue, first in a hypertonic solution and then in a hypotonic solution;
incubating the tissue in a solution comprising an emulsifier; and
sterilizing the tissue.

2. The method of claim 1, further comprising after sterilizing the tissue, lyophilizing the tissue.

3. The method of claim 2, further comprising the step of reconstituting the tissue into a gel.

4. The method of claim 2, further comprising mixing one or both of a biological polymer composition and a synthetic polymer composition with the CNS ECM gel.

5. The method of claim 1 in which one or more of the steps of thawing, digesting, incubating the tissue in a solution comprising a detergent, osmotically shocking and incubating the tissue in a solution comprising an emulsifier is performed while agitating the sample.

6. The method to of claim 1, wherein the CNS tissue is brain tissue.

7. The method of claim 1, wherein the CNS tissue is spinal cord tissue.

8. The method of claim 1, wherein dura mater is removed from the CNS tissue prior to digesting the tissue with a protease.

9. The method of claim 1, wherein protease is trypsin.

10. The method of claim 1, wherein the detergent is a non-ionic detergent.

11. The method of claim 1, wherein the detergent is a 4-octylphenol polyethoxylate.

12. The method claim 1, wherein the emulsifier is a deoxycholate solution.

13. The method of claim 1, further comprising mixing one or both of a biological polymer composition and a synthetic polymer composition with the CNS ECM material.

14. The method of claim 1, in which the tissue is thawed in one of water, PBS or normal saline.

15. The method of claim 1, further comprising drying the CNS ECM material.

16. The method of claim 15, further comprising comminuting the CNS ECM material into a powder.

17. The method of claim 15, further comprising comminuting the CNS ECM material into a powder of a particle size of 840 ÿm or less.

18. The method of claim 15, further comprising re-hydrating the dried CNS ECM material.

19. A product produced by the process of claim 1.

20. The product of claim 19, wherein the ECM is at a concentration between about 1 mg/ml and 200 mg/ml.

21. The product of claim 19, wherein the ECM is at a concentration between about 1 mg/ml and 50 mg/ml.

22. The product of claim 19, wherein the ECM is at a concentration between about 2 mg/ml and 20 mg/ml.

23. A method of treatment for CNS injury or disease in a mammal, comprising injecting the product of claim 19 into a site of CNS injury or disease in the mammal.

24. A device comprising a product produced by the process of claim 1.


SEQUENCE LISTING (7 sequences)

SEQ ID NO: 1 - protein, 4 aa, Homo sapiens
RGDS

SEQ ID NO: 2 - protein, 4 aa, Homo sapiens
REDV

SEQ ID NO: 3 - protein, 5 aa, Homo sapiens
PHSRN

SEQ ID NO: 4 - protein, 5 aa, Homo sapiens
KNEED

SEQ ID NO: 5 - protein, 5 aa, Homo sapiens
YIGSR

SEQ ID NO: 6 - protein, 5 aa, Homo sapiens
IKVAV

SEQ ID NO: 7 - protein, 4 aa, Homo sapiens
DGEA